(19) United States
(12) Patent Application Publication
(10) Pub. No.: US 2015/0093821 A1
(43) Pub. Date: Apr. 2, 2015

(54) BIODEGRADABLE POLYURETHANES AND USE THEREOF

(71) Applicants: CARNEGIE MELLON UNIVERSITY, Pittsburgh, PA (US); University of Pittsburgh -- Of The Commonwealth System of Higher Education, Pittsburgh, PA (US)

(72) Inventors: Eric J. Beckman, Aspinwall, PA (US); Jeffrey O. Hollinger, Gibsonia, PA (US); Bruce A. Doll, Wexford, PA (US); Scott A. Guelcher, Thompson Station, TN (US); Jianying Zhang, Pittsburgh, PA (US)

(21) Appl. No.: 14/511,370

(22) Filed: Oct. 10, 2014

Related U.S. Application Data
(63) Continuation of application No. 10/759,904, filed on Jan. 16, 2004, now abandoned.
(60) Provisional application No. 60/440,544, filed on Jan. 16, 2003.

(51) Int. Cl. A61L 27/18; C12N 5/077; A61L 27/38; A61L 27/54; A61L 27/58

(52) U.S. Cl. CPC A61L 27/18; A61L 27/54; A61L 27/58; A61L 27/3821; C12N 5/0654; A61L 2300/43; A61L 2430/02; C12N 2533/40
USPC 435/366; 527/301

(57) ABSTRACT

A biodegradable and biocompatible polyurethane composition synthesized by reacting isocyanate groups of at least one multifunctional isocyanate compound with at least one bioactive agent having at least one reactive group —X which is a hydroxyl group (—OH) or an amine group (—NH₂). The polyurethane composition is biodegradable within a living organism to biocompatible degradation products including the bioactive agent. Preferably, the released bioactive agent affects at least one of biological activity or chemical activity in the host organism. A biodegradable polyurethane composition includes hard segments and soft segments. Each of the hard segments is preferably derived from a diurea diol or a diester diol and is preferably biodegradable into biomolecule degradation products or into biomolecule degradation products and a biocompatible diol. Another biodegradable polyurethane composition includes hard segments and soft segments. Each of the hard segments is derived from a diurethane diol and is biodegradable into biomolecule degradation products.

### CROSS-REFERENCE TO RELATED APPLICATION

This application claims the benefit of U.S. Provisional Patent Application Ser. No. 60/440,544, entitled BIODEGRADABLE POLYURETHANES AND USE THEREOF IN TISSUE ENGINEERING filed Jan. 16, 2003, the disclosure of which is incorporated herein by reference.

### BACKGROUND OF THE INVENTION

The present invention relates generally to biodegradable polyurethanes and to the use thereof, and particularly to biodegradable polyurethanes for use in tissue engineering.

References set forth herein may facilitate understanding of the present invention or the background of the present invention. Inclusion of a reference herein, however, is not intended to and does not constitute an admission that the reference is available as prior art with respect to the present invention.

Synthetic biodegradable polymers hold promise in a number of fields, including use as scaffolds in tissue engineering. Bone repair, for example, is an attractive and natural target for tissue engineering, as bone regeneration is needed for the therapy of numerous serious clinical indications. Many materials, including autografts, allografts and xenografts, as well as a variety of biomaterials based on ceramics, metals, polymers, and a host of composites thereof, are currently used to repair or replace bone that has been damaged as a result of trauma or disease. The use of allografts and xenografts is limited by the risk of an immunological response and the risk of disease transmission. Autografts are restricted by a limited number of donor sites and are associated with additional trauma resulting from the harvesting of bone tissue as well as the potential for mortality. Synthetic materials thus stand out as a potential solution, being readily available, processable, and modifiable to suit the needs of a given application.

However, many problems persist from the inability to exactly match the properties of natural tissue. Most metals, for example, exhibit mechanical properties far exceeding those of bone, which results in stress shielding and the subsequent weakening of the host bone tissue, thereby making it susceptible to re-fracture. Ceramics, particularly calcium phosphate-based ceramics such as hydroxyapatite (HA), are brittle and difficult to mold into a variety of shapes.

Synthetic biodegradable polymers offer a promising replacement material because of, for example, ease of synthesis, virtually unlimited supply, and the potential of coupling polymer degradation and removal with concurrent tissue regeneration. An important factor to consider when choosing a polymer for biological use is the toxicity of the polymer and the associated degradation products. Degradable materials also must maintain their mechanical integrity for a sufficient period of time to allow the ingrowth of tissue necessary for bone formation. Polymer scaffolds used in bone tissue engineering must also support bone cell attachment and differentiation, as well as stimulate bone cell proliferation, type I collagen, and alkaline phosphatase synthesis.

Polyurethane elastomers have been used in biomedical applications for a number of years. However, most of these applications are in non-degradable devices, such as cardiovascular catheters and infusion pumps. Polyurethane elastomers are, however, susceptible to in vivo degradation via both chemical and enzymatic hydrolysis. Moreover, polyether-based polyurethane elastomers are susceptible to environmental stress-cracking as a result of degradation by enzymes (such as cathepsin B), and considerable research has focused on synthesizing polyurethane elastomers that are not susceptible to stress-cracking. Conventional polyurethane elastomers are typically reaction products of aromatic isocyanates and hexamethylene diamine. For example, U.S. Pat. No. 6,306,177 discloses a method, composition, and apparatus for repairing the site of injured tissue by delivering to the site a curable biomaterial composed of a (1) quasi-prepolymer component comprising the reaction product of an isocyanate and a polyol and (2) a curative component comprising a polyol, chain extender and catalyst. Certain aromatic diisocyanates, such as preferred for use in U.S. Pat. No. 6,306,177, degrade slowly, if at all, and their degradation products include toxic materials such as aromatic diamines. Low molecular weight isocyanates (such as toluene diisocyanate [TDI] and 2,2′-, 2,4′-, and 4,4′-diphenylmethanediisocyanate [MDI]) are volatile, toxic, and highly reactive, thereby making them undesirable for use in vivo.

Zhang and coworkers synthesized biodegradable lysine diisocyanate ethyl ester (LDI)/glucose polyurethane foams proposed for tissue engineering applications. In those studies, NCO-terminated prepolymers were prepared from LDI and glucose. The prepolymers were chain-extended with water to yield biocompatible foams which supported the growth of rabbit bone marrow stromal cells in vitro and were non-immunogenic in vivo. Zhang, J.-Y., Beckman, E. J., Piesco, N. J. & Agarwal, S. A new peptide-based urethane polymer: synthesis, biodegradation, and potential to support cell growth in vitro. Biomaterials 21, 1247-1258 (2000); Zhang, J.-Y. et al. Synthesis, biodegradability, and biocompatibility of lysine diisocyanate-glucose polymers. Tissue Engineering 8, 771-785 (2002).

U.S. Pat. No. 6,221,997 discloses a biodegradable polyurethane formed by reaction of a polyol, a diisocyanate, and a chain extender. The chain extender is the reaction product of a diol with an amino acid that is in such a condition that it can be recognized by a biological agent. In that regard, the amino acid is subject to enzymatic degradation, thereby enabling a degree of control over the degradation of the polyurethane. Amino acid-based or other aliphatic diisocyanates are disclosed as preferred, as the toxicity of the resulting degradation products is less than that of conventional aromatic diisocyanates. Aliphatic diols such as 1,4-cyclohexane dimethanol are disclosed as preferred for the synthesis of the chain extender.

U.S. Pat. No. 6,376,742 discloses a porous scaffold fabricated from a biodegradable polyurethane for the delivery of cells to repair diseased tissue. The components of a biocompatible polymerizable composition including a blowing agent are combined and delivered to the body to form in vivo a porous polymer structure which permits cellular ingrowth. Seed cells can be optionally added to the polymerizable composition. Both aliphatic and aromatic isocyanates are disclosed in the synthesis of the biodegradable polyurethanes. Aliphatic isocyanates are preferred because they do not degrade to potentially toxic aromatic diamines. The incorporation of bioactive species into the scaffold or cell encapsulation is discussed. For example, the use of proteins to mediate the interface between the host and the implant is indicated to be desirable.

Bioactive polymeric materials in which a bioactive material is, for example, adsorbed upon, encapsulated within or otherwise immobilized by a biodegradable polymer and released into an organism upon biodegradation, have recently attracted interest for tissue engineering and other applications. For example, U.S. Pat. No. 6,534,084 a porous foam for the regeneration of tissue comprising contacting cells with a biocompatible foam that has a gradient in composition or microstructure. The structure of the foam is described to be controlled and not random to optimally support cell growth. The foam can optionally be seeded with cells. Therapeutic and bioactive agents can be coated on the polymer foam or incorporated into the polymers used to make the foam.

U.S. Pat. No. 6,409,764 discloses a shell-like device for implantation into the body which is capable of being penetrated by cells. The device establishes a space wherein at least one protein from the transforming growth factor (TGF)-beta family is placed to stimulate the growth of living bone. The (TGF)-beta protein can be incorporated into a carrier such as a biodegradable polymer.

U.S. Pat. No. 5,916,585 discloses a biodegradable material for immobilization of a bioactive species including a hydrophobic biodegradable support member and a polymeric surfactant layer adsorbed to the support member. A bioactive species is immobilized via chemically functions groups of the surfactant polymer or through unreacted chemically functional groups of a crosslinking agent used to crosslink the hydrophilic polymer.

Non-degradable polyurethanes have also been used to immobilize active enzymes. For example, U.S. Pat. No. 6,291,200 describes a sensor for detecting the presence of an analyte including an enzyme and indicator compound incorporated within a polymer. The enzyme can be covalently bound to the polymer, which is preferably a polyurethane. Proteins, which contain many amine and hydroxyl groups, react with isocyanate groups during synthesis of the polymers, thereby forming a polyurethane which contains covalently bound enzymes.

Developing bio-functional polymers for load-bearing applications such as scaffolds for knee-joint meniscus presents a number of additional design and development challenges. Polyurethane elastomers, for example, are generally linear molecules including alternating hard and soft segments, which give the polymers favorable mechanical properties. Depending on the conditions, hard segments in neighboring chains can aggregate (as a result of hydrogen bonding between urea and urethane linkages in the backbone) and form paracrystalline domains, thereby increasing the hardness of the elastomer. By varying the composition, polyurethanes elastomers having a broad range of properties ranging from soft to hard can be prepared.

However, the diisocyanate and chain extender intermediates typically used in the hard segment of conventional polyurethanes are not biocompatible. For example, conventional polyurethanes are often based on MDI, which decomposes to a toxic aromatic diamine as described above.

Paracrystalline biodegradable polyurethanes synthesized from aliphatic diisocyanates have been described by Pennings and co-workers using butane diisocyanate (BDI), an ε-polycaprolactone (PCL) soft segment, and putrescine (butanediamine, BDA) and butanediol (BDO) chain extenders. Spaans, C. J. et al. Solvent-free fabrication of micro-porous polyurethane-amide and polyurethane-urea scaffolds for repair and replacement of the knee-joint meniscus. Biomaterials 21, 2453-2460 (2000); De Groot, J. H., de Vrijer, R., Wildeboer, B. S., Spaans, C. J. & Pennings, A. J. New biomedical polyurethane ureas with high tear strengths. Polymer Bulletin 38, 211-218 (1997); Spaans, C. J., De Groot, J. H., Belgraver, V. W. & Pennings, A. J. A new biomedical polyurethane with a high modulus based on 1,4-butanediisocyanate and e-caprolactone. Journal of Materials Science: Materials in Medicine 9, 675-678 (1998); Spaans, C. J., De Groot, J. H., Dekens, F. G. & Pennings, A. J. High molecular weight polyurethanes and a polyurethane urea based on 1,4-butanediisocyanate. Polymer Bulletin 41, 131-138 (1998); Spaans, C. J., De Groot, J. H., Van der Molen, L. M. & Pennings, A. J. New biodegradable polyurethane-ureas, polyurethane and polyurethane-amide for in-vivo tissue engineering: structure-properties relationships. Polymeric Materials Science and Engineering 85, 61-62 (2001); and European Patent Application No. EP 1308473. The BDO.BDI.BDO elastomers were used to make porous knee meniscus scaffolds using the salt leaching/freeze drying technique. Pennings and co-workers also synthesized elastomers from PCL and BDA using HDI, BDI, and LDI. The BDI- and HDI-(Tₘ=250° C.) based elastomers had high modulus and tensile strength, but the LDI-(Tₘ=91° C.) based elastomer had weak mechanical properties. The soft properties of the LDI elastomer can be explained by the structure of LDI, particularly its asymmetry, odd number of carbon atoms, ethyl ester branch, and relatively low molecular weight.

Woodhouse and co-workers have prepared biodegradable polyurethane scaffolds for soft tissue using lysine methyl ester diisocyanate (LDI), a phenylalanine-based chain extender, and polyethylene glycol (PEG) or polycaprolactone (PCL) diols. See U.S. Pat. No. 6,221,997. The PEG materials were weak, tacky, and amorphous, while the PCL materials were elastomers that under certain conditions were paracrystalline. The phenylalanine-based chain extender was shown to promote degradation due to the cleavage by chymotrypsin of ester bonds adjacent to phenylalanine residues. Fromstein, J. D. & Woodhouse, K. A. Elastomeric biodegradable polyurethane blends for soft tissue applications. Journal of Biomaterials Science Polymer Edition 13, 391-406 (2002). The chain extender was prepared by coupling via Fischer esterification two phenylalanine (Phe) molecules with cyclohexane dimethanol (CHDM). It therefore has two side chains with phenyl groups, making it highly branched and nonlinear. Skarja, G. A. & Woodhouse, K. A. Synthesis and characterization of degradable polyurethane elastomers containing an amino-acid based chain extender. Journal of Biomaterials Science Polymer Edition 9, 271-295 (1998). This rather bulky chain extender makes it difficult for the hard segment to pack into a crystal lattice. The paracrystallinity appears to be from the PCL soft segment and not the hard segment, as evidenced by the melting point of the polymer (Tₘ≅60° C.) compared to that of PCL (Tₘ=43-45° C.). This observation is reinforced by the fact that elastomers could not be prepared from PEO soft segments. The absence of hard segment crystallinity has a significant effect on mechanical properties; the PCL-based polyurethanes had yield points at rather low (<20%) elongations. Skarja, G. A. & Woodhouse, K. A. Structure-property relationships of degradable polyurethane elastomers containing an amino acid-based chain extender. Journal of Applied Polymer Science 75, 1522-1534 (2000).

Suter and co-workers have described biodegradable polyesterurethanes for medical applications such as nerve guide channels. Saad, B. et al. Development of degradable polyesterurethanes for medical applications: In vitro and in vivo evaluations. Journal of Biomedical Materials Research 36, 65-74 (1997); Saad, B., Neuenschwander, P., Uhlschmid, G. K. & Suter, U. W. New versatile, elastomeric, degradable polymeric materials for medicine. International Journal of Biological Macromolecules 25, 292-301 (1999); and Borkenhagen, M., Stoll, R. C., Neuenschwander, P., Suter, U. W. & Aebischer, P. In vivo performance of a new biodegradable polyester urethane system used a nerve guidance channel. Biomaterials 19, 2155-2165 (1998). These materials included a crystallizable macrodiol (α,ω-dihydroxy-oligo[((R)-3-hydroxybutyrate-co-(R)-3-hydroxyvalerate)-block-ethylene glycol, or PHB/HV-diol) and a non-crystallizable macrodiol (α,ω-dihydroxy-poly[ε-caprolactone-block-diethylene glycol-block-ε-caprolactone, or PCL-diol) chain-extended with lysine methyl ester diisocyanate (LDI). The materials were paracrystalline (as a result of the presence of the PHB segment) with melting points below 140° C. The materials were found to be both cell- and tissue-compatible and biodegradable with elastic moduli ranging from 30 MPa to 1200 MPa and degradation times ranging from weeks to years. However, those materials differ significantly from conventional polyurethanes in that the hard segment is composed solely of crystalline PHB rather than a diisocyanate and a chain extender.

Kylmä and Seppälä prepared polyesterurethanes using a similar procedure to that of Suter. Kylma, J. & Seppala, J. V. Synthesis and characterization of a biodegradable thermoplastic poly(ester-urethane) elastomer. Macromolecules 30, 2876-2882 (1997). Lactic acid and caprolactone were copolymerized, capped with butanediol, and chain extended with 1,6-hexamethylene diisocyanate (HDI). The polyurethanes produced by this synthesis were all amorphous. Gorna and Gogolewski studied the degradation and calcification of polyesterurethanes prepared using a procedure similar to that of Kylmä and Seppälä. Gorna, K. & Gogolewski, S. Biodegradable polyurethanes for implants. II. In vitro degradation and calcification of materials from poly(epsilon-caprolactone)-poly(ethylene oxide) diols and various chain extenders. Journal of Biomedical Materials Research 60, 592-606 (2002).

U.S. Pat. No. 6,210,441 describes a linear block polymer comprising urea and urethane groups with ester groups at such a distance from each other such that small fragments result from biodegradation that can be excreted from the human body. The fragments, which are generated upon hydrolysis of the ester groups, may, however, include potentially harmful moieties (for example, groups derived from certain diisocyanates such as MDI which can degrade into potentially harmful diamines), thereby posing a potential hazard should the fragments further degrade. Published PCT Application No. WO 02/053616 describes a polyurethane containing diamine chain extenders. The chain extenders can be prepared from amino acids esterified with diacids or with diols. Diisocyanates described as suitable for use in synthesizing those polyurethanes included MDI, HDI, H₁₂MDI, LDI, IPDI, and TDI. Like U.S. Pat. No. 6,210,441, the polymer of WO 02/053616 degrade into relatively small fragments that can be excreted from the human body or metabolized.

Although substantial effort has been expended in developing biodegradable polyurethanes having various properties, it remains desirable to develop improved, biodegradable polyurethane compositions for use as bio-functional polymers.

### SUMMARY OF THE INVENTION

In one aspect, the present invention provides a bioactive, biodegradable and biocompatible polyurethane composition synthesized by reacting isocyanate groups of at least one multifunctional isocyanate compound with at least one bioactive agent having at least one reactive group —X which is a hydroxyl group (—OH) or an amine group (—NH₂). The polyurethane composition is biodegradable within a living organism to biocompatible degradation products including the bioactive agent. Preferably, the released bioactive agent affects at least one of biological activity or chemical activity in the host organism.

The multifunctional isocyanate compound can, for example, be formed via conversion of amine groups of a biocompatible compound having at least two amine groups to isocyanate groups. In several embodiment, the bioactive agent has at least two reactive groups —X and —X¹ which are independently the same or different a hydroxyl group (—OH) or an amine group (—NH₂). The multifunctional isocyanate compound can also be reacted with at least one biocompatible polyol compound having at least two reactive groups —X² and —X³ which are independently the same of different hydroxyl group (—OH) or an amine group (—NH₂). The multifunctional isocyanate can further be reacted with at least one biocompatible chain extender, wherein the chain extender is water or a compound having at least two reactive groups —X⁴ and —X⁵ which are independently the same of different hydroxyl group (—OH) or an amine group (—NH₂).

In one embodiment, the multifunctional isocyanate compound, the bioactive agent and the polyol compound are reacted to form a prepolymer. The prepolymer is further reacted with at least one biocompatible chain extender, wherein the chain extender is water or a compound having at least two reactive groups —X⁴ and —X⁵ defined as set forth above.

In another embodiment, the multifunctional isocyanate compound is a prepolymer formed by the reaction of a multifunctional isocyanate precursor and at least one biocompatible polyol compound. The polyol compound has at least two reactive groups —X² and —X³ defined as set forth above. The multifunction isocyanate precursor is, for example, formed via conversion of amine groups of a biocompatible compound having at least two amine groups to isocyanate groups. The prepolymer can be contacted with the bioactive agent. In one embodiment, the bioactive compound is in a solution with at least one biocompatible chain extender, wherein the chain extender is water or a compound having at least two reactive groups —X⁴ and —X⁵ defined as set forth above. The bioactive agent can, for example, be an enzyme, an organic catalysts a ribozyme, an organometallic, a protein, a glycoprotein, a lipoprotein, a peptide, a polyamino acid, an antibody, a nucleic acid, a steroidal molecule, an antibiotic, an antiviral, an antimycotic, an anticancer agent, an immunosuppressant, a cytokine, a carbohydrate, an oleophobic, a lipid, an extracellular matrix, a component of an extracellular matrix, a chemotherapeutic agent, an anti-rejection agent, an analgesic agent, an anti-inflammatory agent, a hormone, a virus, a viral vector, a vireno, or a prion.

The multifunctional isocyanate precursor can be an aliphatic multifunctional isocyanate. Preferably, the multifunctional isocyanate precursor is derived from a biomolecule (for example, an amino acid). The polyol compound can also a biomolecule or be derived from a biomolecule. For example, the polyol compound can be a hydroxylated biomolecule. Likewise, the chain extender can a biomolecule or be derived from a biomolecule. In one embodiment, the chain extender is water.

Preferably, the bioactive agent has amine and/or hydroxyl functionality greater than or equal to two. The bioactive agent preferably a molecular weight within the range of approximately 10 to approximately 1,000,000 g/mol. In one embodiment, the bioactive species has inductive capacity for restoration of tissue.

In one embodiment, the polyurethane is a porous foam. Foaming can, for example, be induced using water as a chain extender. The diameter of the pores can, for example, be in the range of approximately 50 μm to approximately 500 μm.

A prepolymer for use in synthesizing the bioactive polyurethanes of the present invention preferably has a free isocyanate content of 1-32 wt-%. The prepolymer can, for example, be synthesized at an NCO:OH equivalent ratio greater than unity. In one embodiment, the prepolymer is synthesized at an NCO:OH equivalent ratio in the range of approximately 1 to approximately 2.

The reactions to synthesize the bioactive, biocompatible and biodegradable polyurethanes of the present invention can proceed with a catalyst or without a catalyst.

In another aspect, the present invention provides a method for the synthesis of a biodegradable, biocompatible, and bioactive polyurethane composition including the step: reacting isocyanate groups of at least one multifunctional isocyanate compound with at least one bioactive agent having at least one reactive group —X which is a hydroxyl group (—OH) or an amine group (—NH₂), the polyurethane composition being biodegradable within a living organism to biocompatible degradation products including the bioactive agent, the released bioactive agent affecting at least one of biological activity or chemical activity in the host organism.

In a further aspect, the present invention provides a method of synthesizing a bone tissue engineering scaffold including the steps of:

coating a biodegradable and bioactive polyurethane polymer with human osteoblastic precursor cells, the polymer being synthesized by reacting isocyanate groups of at least one multifunctional isocyanate compound with at least one bioactive agent having at least one reactive group —X which is a hydroxyl group (—OH) or an amine group (—NH₂), the polyurethane being biodegradable within a living organism to biocompatible degradation products including the bioactive agent, the released bioactive agent affecting at least one of biological activity or chemical activity in the host organism; and

culturing the osteoblastic precursor cells under conditions suitable to promote cell growth.

In one embodiment, prior to coating the osteoblastic precursor cells upon the biocompatible, biodegradable polyurethane, the polyurethane is synthesized by the steps:

reacting at least one multifunctional isocyanate precursor compound with at least one biocompatible polyol compound, the polyol compound having at least two reactive groups —X² and —X³ which are independently the same of different hydroxyl group (—OH) or an amine group (—NH₂) to form the multifunctional isocyanate compound, which is an isocyanate-terminated prepolymer, the multifunction isocyanate precursor compound being formed via conversion of amine groups of a biocompatible compound having at least two amine groups to isocyanate groups;
sterilizing the isocyanate-terminated prepolymer,
dissolving the bioactive agent in at least one sterile chain extender, the bioactive agent having at least two reactive groups —X and —X¹ which are independently the same or different a hydroxyl group (—OH) or an amine group (—NH₂), the chain extender having at least two reactive groups —X⁴ and —X⁵ which are independently the same of different hydroxyl group (—OH) or an amine group (—NH₂); and
contacting the isocyanate-terminated prepolymer with the solution of the bioactive agent and the chain extender to form a polyurethane bone tissue engineering scaffold.

As set forth above, the prepolymer preferably has a free isocyanate content of 1-32 wt-%. In one embodiment, the prepolymer is synthesized at an NCO:OH equivalent ratio greater than unity. In another embodiment, the prepolymer is synthesized at an NCO:OH equivalent ratio in the range of approximately 1 to approximately 2.

In one embodiment, the chain extender is water to create a foamed polyurethane. The bioactive agent can, for example, have a therapeutic or other type of effect in the organism upon release. Examples of suitable bioactive agents are as set forth above. In one embodiment, the bioactive agent is a growth factor. Other suitable bioactive agents include ascorbic acid, dexamethasone and β-glycerolphosphate.

As described above, the multifunctional isocyanate precursor compound can, for example, be an aliphatic multifunctional isocyanate. The multifunctional amine compound from which the multifunctional isocyanate precursor compound is derived can be a biomolecule or a biocompatible derivative of a biomolecule. For example, the multifunctional amine compound can be an amino acid or a biocompatible derivative of an amino acid. For example, the multifunctional amine compound can be lysine, lysine ethyl ester, lysine methyl ester, putrescine, arginine, glutamine or histidine. The multifunctional amine compound can also be a biocompatible diester diamine derived from biomolecules or from a biomolecule and a biocompatible diol.

The polyol compound can also be a biomolecule or a biocompatible derivative of a biomolecule. In one embodiment, the polyol compound is a hydroxylated biomolecule. Examples of suitable polyols for the bioactive, biocompatible and biodegradable polyurethanes include, but are not limited to, a polyether, polytetramethylene etherglycol, polypropylene oxide glycol, polyethylene oxide glycol, a polyester, polycaprolactone, a polycarbonate, a saccharide, a polysaccharide, castor oil, a hydroxylated fatty acid, a hydroxylated triglyceride, or a hydroxylated phospholipids. In one embodiment, a chain extender, which is a biomolecule, is reacted with the prepolymer.

In another aspect, the present invention provides a method of delivering a bioactive agent into an organism including the steps:

injecting at least on multifunctional isocyanate compound into the organism;
injecting at least one bioactive agent into the organism, having at least two reactive groups —X and —X¹ which are, independently the same or different, a hydroxyl group (—OH) or an amine group (—NH₂), the polyurethane composition being biodegradable within a living organism to biocompatible degradation products including the bioactive agent; and
contacting multifunctional isocyanate compound with the bioactive agent to react the isocyanate groups of the multifunctional isocyanate compound with the bioactive agent.

In one embodiment, the method can further include the steps:

injecting at least one biocompatible polyol compound into the organism, the polyol compound having at least two reactive groups —X² and —X³ which are independently the same of different hydroxyl group (—OH) or an amine group (—NH₂);
contacting the polyol compound with the multifunctional isocyanate compound within the organism to react the polyol compound with the multifunctional isocyanate compound.

In another embodiment the method of can further including the steps:
injecting at least one biocompatible chain extender into the organism, wherein the chain extender is water or a compound having at least two reactive groups —X⁴ and —X⁵ defined as set forth above. The multifunctional isocyanate compound, the bioactive agent and the polyol compound can, for example, be reacted to form a prepolymer, which can be injected separately from the biocompatible chain extender.

Preferably, water is used as a chain extender to induce foaming. To enhance the reaction rate/foaming, a second chain extender compound wherein —X⁴ and X⁵ are amine groups can be used in addition to water.

In one embodiment, the multifunctional isocyanate compound can be a prepolymer formed by the reaction of a multifunctional isocyanate precursor and the biocompatible polyol compound, wherein the multifunction isocyanate precursor is, for example, formed via conversion of amine groups of a biocompatible compound having at least two amine groups to isocyanate groups.

The prepolymer can be injected separately from the bioactive agent. For example, the bioactive compound can be in a solution with at least one biocompatible chain extender, the chain extender having at least two reactive groups —X⁴ and —X⁵ which are independently the same of different hydroxyl group (—OH) or an amine group (—NH₂). As discussed above, water is preferably used as a chain extender to induce foaming Once again, another chain extender wherein the groups —X⁴ and —X⁵ are amine groups can be used to enhance the rate of reaction.

In another alternative embodiment, the bioactive agent, the biocompatible polyol and the biocompatible chain extender are injected as a mixture and the multifunctional isocyanate compound is injected separately.

In still another aspect, the present invention provides an implant for insertion into an organism. The implant is formed external to the organism and subsequently placed into the organism. The implant is formed by reacting isocyanate groups of at least one multifunctional isocyanate compound with at least one bioactive agent having at least one reactive group —X which is a hydroxyl group (—OH) or an amine group (—NH₂) as set forth above. The polyurethane composition is biodegradable within a living organism to biocompatible degradation products including the bioactive agent. The released bioactive agent affects at least one of biological activity or chemical activity in the host organism.

The bioactive, biocompatible and biodegradable polyurethanes of the present invention can be synthesized with a wide variety of physiochemical characteristics and morphologies. Moreover, unlike many previous bioactive polymers, the bioactive agents of the bioactive, biocompatible and biodegradable polyurethanes of the present invention can be distributed generally homogeneously within the polyurethane matrix, providing a gradual and generally consistent release of the bioactive species upon degradation.

In another embodiment, the present invention provides a biodegradable polyurethane composition including hard segments and soft segments. Each of the hard segments is preferably derived from a diurea diol or a diester diol and is preferably biodegradable into biomolecule degradation products or into biomolecule degradation products and a biocompatible diol. In one embodiment, the hard segments include groups derived from at least one diisocyanate which results in a diamine biomolecule degradation product upon biodegradation of the polyurethane. The diisocyanate groups of the hard segment can, for example, be derived from butane diisocyanate, lysine diisocyanate, lysine ethyl ester diisocyanate or lysine methyl ester diisocyanate. The segmented polyurethanes of the present invention can be synthesized in reactions with or without catalysts.

The hard segments preferably further include at least one group derived from a chain extender. In one embodiment, the chain extender is a diurea diol or a diester diamine. In the case that the chain extender is a diurea diol, the diurea diol can be formed by the reaction of one molecule of a biocompatible diisocyanate with two molecules of a multifunctional biomolecule having a hydroxy group and an amine group. The multifunctional biomolecule can, for example, be tyramine, tyrosine ethyl ester, tyrosine methyl ester, serine ethyl ester, serine methyl ester or pyridoxamine.

In the case that the chain extender is a diester diamine, the diester diamine can, for example, be formed by reacting one molecule of a diacid biomolecule with two molecules of a multifunctional biomolecule having a hydroxy group and an amine group Amine groups in this and other reactions of the present invention can be protected to prevent undesirable reactions. Suitable protective groups for amino groups include, but are not limited to, tert-butyloxycarbonyl, formyl, acetyl, benzyl, p-methoxybenzyloxycarbonyl, trityl. Other suitable protecting groups as known to those skilled in the art are disclosed in Greene, T., Wuts, P. G. M., Protective Groups in Organic Synthesis, Wiley (1991), the disclosure of which is incorporated herein by reference. In general, protecting groups used in the methods of the present invention are preferably chosen such that they can be selectively removed without affecting the other substituents on the reaction product. The diacid biomolecule can, for example, be succinic acid or adipic acid. The multifunctional biomolecule reacted with the diacid biomolecule can, for example, be tyramine, tyrosine ethyl ester, tyrosine methyl ester, serine ethyl ester, serine methyl ester or pyridoxamine.

In another embodiment, in which the chain extender is a diester diamine, the diester diamine can be formed by reacting one molecule of a biocompatible diol with two molecules of a multifunctional biomoleule having an amine group and a carboxylic acid group or an ester group. The amine group can be protected. The multifunctional biomolecule can, for example, be p-aminobenzoic acid, ethyl p-aminobenzoate, glycine, glycine ethyl ester or glycine methyl ester. The biocompatible diol can, for example, be butanediol.

In one embodiment, the diurea diol of the chain extender has the formula:

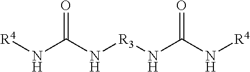

wherein R³ is

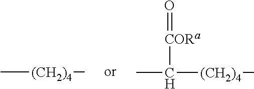

wherein R⁴ is

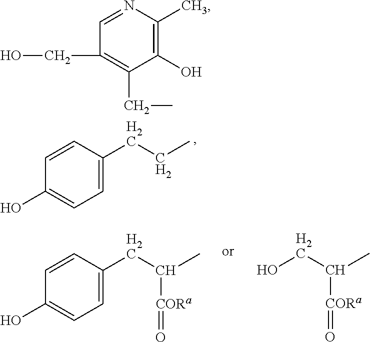

and wherein Ra is —CH₃ or —CH₂CH₃.

In another embodiment, the diester diamine of the chain extender has the formula:

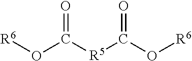

wherein R⁵ is

—(CH₂)ₙ—

wherein n is 2 or 4, wherein R⁶ is

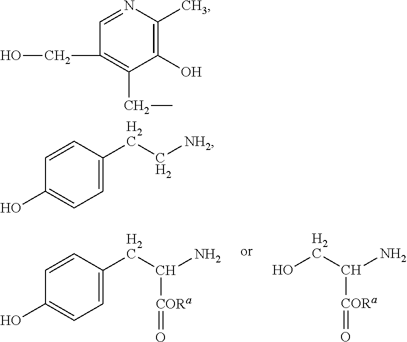

and wherein R^{a} is —CH₃ or —CH₂CH₃.

The diester diamine of the chain extender can also have the formula:

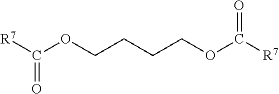

wherein R⁷

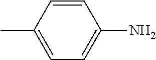

### or —CH₂—NH₂

In a further aspect, the present invention provides an implant for use in a living organism. The implant includes a biodegradable polyurethane composition including hard segments and soft segments. Each of the hard segments is derived from a diurea diol or a diester diamine and is biodegradable into biomolecule degradation products or into biomolecule degradation products and a biocompatible diol. As discussed above, the hard segments are derived from the reaction of a diurea diol or a diester diamine with a diisocyanate preferably derived from a biomolecule.

In another aspect, the present invention provides a biodegradable polyurethane composition including hard segments and soft segments. Each of the hard segments is derived from a diurethane diol and is biodegradable into biomolecule degradation products. The hard segments preferably include groups derived from at least one diisocyanate which results in a diamine biomolecule degradation product upon biodegradation of the polyurethane. The diisocyanate groups of the hard segment can, for example, be derived from butane diisocyanate, lysine diisocyanate, lysine ethyl ester diisocyanate or lysine methyl ester diisocyanate.

The hard segments further include at least one group derived from a chain extender. The chain extender is preferably a diurethane diol. The diurethane diol chain extender can, for example, be formed by reacting one molecule of a biocompatible diisocyanate with two molecules of a multifunctional biomolecule having two hydroxy groups. The multifunctional biomolecule can, for example, be glyceraldehyde, dihydroxyacetone or pyridoxine.

In one embodiment, the diurethane diol has the formula:

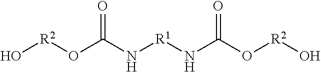

wherein R¹ is

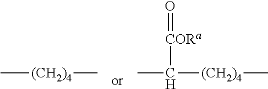

and
and wherein R² is

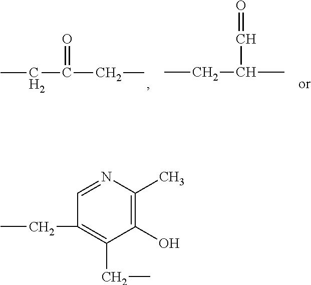

and wherein R^{a} is —CH₃ or —CH₂CH₃.

In a further aspect, the present invention provides an implant for use in a living organism. The implant includes a biodegradable polyurethane composition including hard segments and soft segments. Each of the hard segments is derived from a diurethane diol and is biodegradable into biomolecule degradation products.

In another aspect, the present invention provides a composition having the formula:

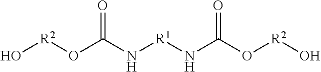

wherein R¹ is

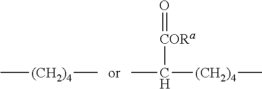

and wherein R² is

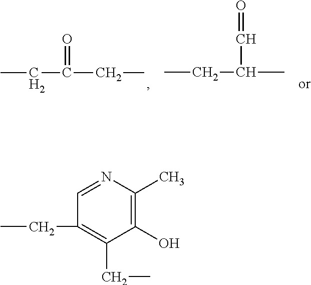

and wherein R^{a} is —CH₃ or —CH₂CH₃.

In another aspect the present invention provides composition having the formula:

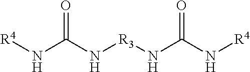

wherein R³ is

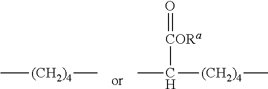

wherein R⁴ is

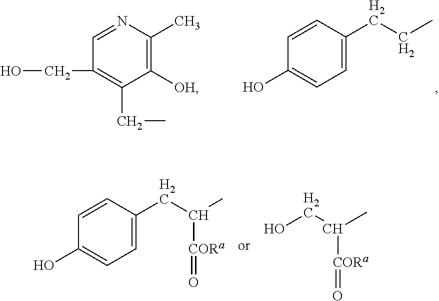

and wherein R^{a} is —CH₃ or —CH₂CH₃.

In still another aspect, the present invention provides a composition having the formula:

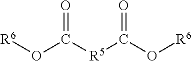

wherein R⁵ is

—(CH₂)ₙ—

wherein n is 2 or 4, and wherein R⁶ is

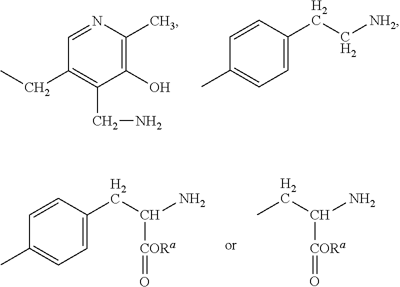

wherein R^{a} is —CH₃ or —CH₂CH₃.

In another aspect, the present invention provides a composition having the formula:

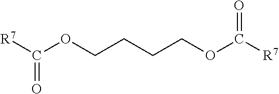

wherein R⁷ is

—CH₂—NH₂.

In a further aspect, the present invention provides a composition having the formula:

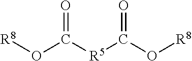

wherein R⁵ is

—(CH₂)ₙ—

wherein n is 2 or 4, wherein R⁸ is

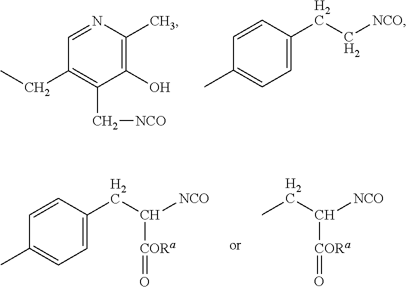

and wherein R^{a} is —CH₃ or —CH₂CH₃.

In still a further aspect, the present invention provides a composition having the formula:

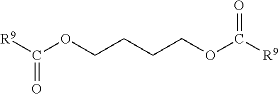

wherein R⁹ is

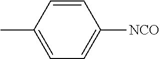

### or —CH₂—NCO

In the reactions to synthesize the bioactive polyurethanes of the present invention or the segmented polyurethanes of the present invention, when a reactant exists in optically isomeric form (for example, amino acids such as lysine, tyrosine and serine), the reactant can be used in racemic form, optically enriched form or optically pure form. For many amino acids that exist as optical isomers, the L-isomer is the most readily available.

### BRIEF DESCRIPTION OF THE DRAWINGS

FIG. 1A illustrates a study of the degradation of an LDI-glycerol-PEG-ascorbic acid polymer of the present invention in aqueous solution with or without fetal bovine serum and sets forth the concentration of lysine released from the LDI-glycerol-PEG-ascorbic acid polymer.

FIG. 1B illustrates a study of the degradation of an LDI-glycerol-PEG-ascorbic acid polymer of the present invention in aqueous solution and sets forth the concentration of glycerol released from the LDI-glycerol-PEG-ascorbic acid polymer.

FIG. 1C illustrates a study of the degradation of an LDI-glycerol-PEG-ascorbic acid polymer of the present invention in aqueous solution and sets forth the concentration of ascorbic acid released from the LDI-glycerol-PEG-ascorbic acid polymer.

FIG. 1D illustrates a study of the effect of degradation products of an LDI-glycerol-PEG-AA polymer of the present invention on the pH of the degradation system.

FIG. 2A illustrates a study of the effect of ethanol on a green fluorescent protein-transgenic mouse bone marrow cells (GFP-MBMC) cultured for 14 days.

FIG. 2B illustrates a study of the concentration of ethanol released from an LDI-glycerol-PEG-ascorbic acid polymer of the present invention in PBS at 37° C. over a period of 60 days.

FIG. 3A illustrates a study of the effect of ascorbic acid-containing polyurethane-urea polymer of the present invention on the cell proliferation.

FIG. 3B illustrates a study of the effect of ascorbic acid-containing polyurethane-urea polymer of the present invention on the alkaline phosphatase activity of bone cells, wherein GFP-MBMC was cultured in the medium without AA (Group 1), in the medium with 30 μg/ml AA (Group 2), on LDI-glycerol-PEG scaffold (Group 3), and on LDI-glycerol-PEG-AA scaffold (Group 4), respectively.

FIG. 4A sets forth a comparison of mRNA expressions of collagen type I and TGF-β1 in GFP-MBMC under the four sets of culture conditions for 14 days; Lane 1: GFP-MBMC grown in the medium without AA (Group 1); Lane 2: GFP-MBMC grown in the medium with 30 μg/ml of ascorbic acid (Group 2); Lane 3: GFP-MBMC grown on the LDI-glycerol-PEG scaffold (Group 3); and Lane 4: GFP-MBMC grown on the LDI-glycerol-PEG-AA scaffold (Group 4).

FIG. 4B illustrates a study of total collagen type I determined on GFP-MBMC after 14 days culture by Sirius Red F3B in GFP-MBMC grown in the medium without AA (Group 1); in the medium with 30 μg/ml of ascorbic acid (Group 2); in LDI-glycerol-PEG scaffold (Group 3) and in LDI-glycerol-PEG-AA scaffold (Group 4).

FIG. 5 illustrates a study of alkaline phosphatase activity for polymer foams synthesized with LDI, glucose and PEG only (DMEM); with LDI, glucose, PEG and β-glycerophosphate (β-GP); with LDI, glucose, PEG and dexamethasone (Dex); with LDI, glucose, PEG and ascorbic acid (Vc); with LDI, glucose, PEG, ascorbic acid and β-glycerophosphate (Vc+β-GP); with LDI, glucose, PEG, ascorbic acid and dexamethasone (Vc+Dex); and with LDI, glucose, PEG, ascorbic acid, β-glycerophosphate and dexamethasone (Vc+Dex+β-GP).

FIG. 6 illustrates a study of cell proliferation for cells cultured over a period of 14 days with Dulbecoo's modified Eagle's medium only (DMEM); with 100 nM dexamethasone (Dex); in the polymer foam synthesized by LDI, glucose, PEG 400 and cultured with DMEM only (Foam); and with the polymer foam synthesized by LDI, glucose and PEG and dexamethasone (Foam-Dex).

FIG. 7 illustrates a comparison of the release of Runx2-pIRESneo plasmid from a dry polymer scaffold and from a wet polymer scaffold.

FIG. 8 illustrates the synthesis of a segmented polyurethane via the prepolymer route.

FIG. 9A illustrates natural metabolites with diol functionality, which yield urethane diols when coupled with a diisocyanate.

FIG. 9B illustrates natural metabolites with amine and hydroxy functionality, which yield urea diols when coupled with a diisocyanate.

FIG. 10A illustrates the structures of butane diisocyanate and L-lysine ethyl ester diisocyanate.

FIG. 10B illustrates the structures of hexamethylene diisocyanate and 4,4′-methylenebis(phenylisocyanate).

FIG. 11A illustrates an embodiment of the structure of a diurethane diol of the present invention.

FIG. 11B illustrates an embodiment of the structure of a diurea diol of the present invention.

FIG. 12A illustrates the preparation of diester diamines by the coupling of two molecules with both hydroxyl and amine functionality with one molecule of succinic acid by the Fischer esterification reaction.

FIG. 12B illustrates the preparation of diester diamines by coupling two molecules of a natural metabolite having carboxylic acid and amine functionality with one molecule of a biocompatible diol.

FIG. 13 illustrates p-aminobenzoic acid and glycine, natural metabolite with carboxylic acid and amine functionality.

FIG. 14 illustrates a standard optical density curve at 550 nm for collagen type I from calf skin.

### DETAILED DESCRIPTION OF THE INVENTION

Bioactive, Biocompatible and Biodegradable Polyurethanes

Biocompatible materials have the ability to perform within a host orgasm without causing inappropriate host responses including, but not limited to, excessive inflammation, excessive injury or excessive death of surrounding tissue due to cytotoxicity. See, for example, Remes, A. and Williams, D. F. Immune response in biocompatibility, Biomaterials, 13:11, 731-43 (1992). In general, the term “biocompatible” as used herein, refers to materials that do not produce any substantial adverse effect within an organism (for example, by causing or inducing excessive inflammation, excessive cytoxicity or other excessive adverse host responses.). The term “biodegradation” as used herein refers to the breakdown of a material mediated by a biological system. See, for example, Remes, A. and Williams, D. F. Immune response in biocompatibility, Biomaterials, 13:11, 731-43 (1992). Biodegradation of the polyurethanes of the present invention can, for example, occur by chemical and/or enzymatic hydrolysis. The polyurethanes of the present invention are both biodegradable and biocompatible. In that regard, the polymers of the present invention in vivo are biocompatible and biodegrade to biocompatible components without a substantial adverse tissue response.

Biocompatible and biodegradable polyisocyanates are preferably used in the synthesis of the polyurethanes of the present invention. Aliphatic polyisocyanates, such as hexamethylene diisocyanate (HDI) are preferred over conventional aromatic polyisocyanates such as MDI and TDI. Aliphatic polyisocyanates degrade to aliphatic diamines that are less toxic than the aromatic diamines which are the degradation products of such conventional aromatic polyisocyanates. Polyisocyanates that are prepared from or derived from biomolecules (including aromatic biomolecules) having multiple amine functionality are preferred. As used herein, the term “biomolecule” refers to a molecule that is commonly found in living cells and tissues. The biodegradation products of polyisocyanates derived from biomolecules are the biomolecules from which they were prepared. For example, lysine diisocyanate (as well as its ethyl ester and methyl ester, collectively LDI) can be prepared from lysine, an amino acid, and butane diisocyanate (BDI) can be prepared from putrescine (1,4-aminobutane), a molecule essential to cell metabolic processes. Polyisocyanates can also be prepared from polyamines by phosgenation. Additional examples of preferred polyisocyanates for use in the bioactive polyurethanes present invention include, but are not limited to, arginine isocyanate, glutamine isocyanate, and histidine isocyanate.

As used herein, the term “polyol” refers to a reactive molecule which contains at least two functional groups that are capable of reacting with an isocyanate group. Most polyols suitable for use in the bioactive, biocompatible and biodegradable polyurethanes of the present invention are amine- and/or hydroxyl-terminated compounds and include, but are not limited to, polyether polyols (such as polyethylene glycol (PEG or PEO), polytetramethylene etherglycol (PTMEG), polypropylene oxide glycol (PPO)); amine-terminated polyethers; polyester polyols (such as polybutylene adipate, caprolactone polyesters, castor oil); and polycarbonates (such as poly(1,6-hexanediol) carbonate). Preferred polyols for use in the bioactive, biocompatible and biodegradable polyurethanes of the present invention include biocompatible and biodegradable polyols such as, for example, lactone-based polyesters (such as poly(ε-caprolactone)) and polyethylene glycol. Particularly preferred polyols for use in the present invention include, but are not limited to: (1) biomolecules having multiple hydroxyl or amine functionality, such as glucose, polysaccharides, and castor oil; and (2) biomolecules (such as fatty acids, triglycerides, and phospholipids) that have been hydroxylated by known chemical synthesis techniques to yield polyols. In preferred embodiments, the polyol degradation products of the polyurethanes are the biomolecules from which they were prepared.

In several embodiments of the present invention, bioactive, biocompatible and biodegradable polyurethanes are provided in which a bioactive agent, molecule or compound is released upon degradation. As used herein, the term “bioactive agent, molecule or compound” refers generally to an agent, a molecule, or a compound that affects biological or chemical events in a host (for example, by inducing, modulating, activating, or inhibiting such biological or chemical events). In the present invention, bioactive polyurethanes are prepared by incorporating bioactive agents into the polymer via covalent bonds resulting from reaction of the biological agents with isocyanate groups during the polymerization process. Bioactive agents suitable for use in the present invention have at least one, and preferably two or more, amine and/or hydroxyl groups that can react with an isocyanate group, thereby incorporating them into the polyurethane. As the polyurethane degrades, the bioactive agents are released and are free to elicit or modulate biological activity. Bioactive agents may be synthetic molecules, biomolecules or multimolecular entities and include, but are not limited to, enzymes, organic catalysts, ribozymes, organometallics, proteins, glycoproteins, peptides, polyamino acids, antibodies, nucleic acids, steroidal molecules, antibiotics, antivirals, antimycotics, anticancer agents, antirejection agents, immunosuppressants, cytokines, carbohydrates (for example, saccharides, polysaccharide, starch etc.), oleophobics, lipids, extracellular matrix and/or its individual components, pharmaceuticals, chemotherapeutics and therapeutics. Cells and non-cellular biological entities, such as viruses, virenos, virus vectors, and prions can also be bioactive agents for use in the present invention. Bioactive molecules that have a formula weight ranging from 50 to 1,000,000 daltons are preferred. Bioactive molecules that have osteogenic properties, such as bone morphogenetic proteins, are, for example, preferred bioactive agents in bone scaffolds of the present invention. Other examples of bioactive molecules suitable for use in the present invention (and particularly useful in bone scaffolds) include ascorbic acid, dexamethasone and β-glycerolphosphate.

The polyurethanes of the present invention can be used to produce articles having various physiochemical properties and morphologies including, for example, flexible foams, rigid foams, elastomers, coatings, adhesives, and sealants. The properties of the polyurethanes of the present invention are controlled by choice of the raw materials and their relative concentrations. For example, thermoplastic elastomers are characterized by a low degree of cross-linking and are typically segmented polymers, consisting of alternating hard (diisocyanate and chain extender) and soft (polyol) segments. Thermoplastic elastomers are formed from the reaction of diisocyanates with long-chain diols and short-chain diol or diamine chain extenders. In contrast to the flexible elastomers, rigid polyurethanes can be formed from stiff (e.g., short chain) reactants having a high functionality. If a portion of either the polyisocyanate, the polyol, or the bioactive agent has a functionality greater than two, the resultant polymer will be crosslinked. Such polymers are typically thermosets and are harder and more rigid than thermoplastics. For example, thermoset rigid foams are characterized by extreme cross-linking and chain stiffness and are formed from short-chain polyols and polymeric isocyanates (such as polymeric MDI) that have a functionality greater than two. For bone and other tissue engineering applications (that is, both hard and soft tissue engineering applications), flexible and rigid foams are preferred because they enable the migration and attachment of cells in the polyurethane, which acts as a scaffold for new bone/tissue growth. In a preferred embodiment, the pores in polyurethane foams of the present invention are interconnected (that is, open) and have a diameter ranging from approximately 50 to approximately 500 μm.

Chain extenders preferred for use in the bioactive, biocompatible and biodegradable polyurethanes of the present invention are low-molecular-weight reactants that can significantly affect the properties of the polyurethane. Chain extenders suitable for use in the bioactive, biocompatible and biodegradable polyurethanes of the present invention are hydroxyl- and/or amine-terminated and preferably have a molecular weight ranging from 10 to 500 Daltons and a functionality of at least two. Chain extenders having a functionality greater than two are also referred to as cross-linkers. Thermoplastic elastomers typically employ a short-chain diol or diamine, such as 1,4-butanediol and ethylene diamine Flexible and rigid foams can be produced by using water as a chain extender. The water generates carbon dioxide which acts as a blowing agent. Biomolecules, such as putrescine (1,4-butanediamine) and water, are preferred chain extenders for use in the present invention.

The polyurethanes of the present invention can, for example, be made via a one-shot process, wherein all the reactants are mixed at once, or via a prepolymer process. In the one-shot process, the polyisocyanate, the polyol, the bioactive component, and optionally a chain extender are added to the reaction mixture at the same time. In one embodiment of the prepolymer process, the polyisocyanate and the polyol are reacted at an NCO:OH equivalent ratio greater than unity to yield an NCO-terminated prepolymer. The prepolymer is then reacted with a hydroxyl- and/or amine-terminated chain extender to yield the polyurethane polymer. The prepolymer process generally enables a greater degree of control over the toxicity, sterility, reactivity, structure, properties, and processibility of the polyurethane. In a preferred embodiment, the prepolymer of the present invention has a free isocyanate content ranging from approximately 1 to approximately 32 wt-%. In the prepolymer process, the bioactive molecule can be added during the prepolymer step or during the chain extension step.

In a preferred embodiment, the polyisocyanate and the polyol are reacted at an NCO:OH equivalent ratio greater than unity to yield an NCO-terminated prepolymer which can be sterilized. The prepolymer can then be reacted with a chain extender in which the bioactive component is dissolved. In this embodiment the bioactive component can itself act as a chain extender or cross-linker. In one preferred embodiment, the polyisocyanate and polyol are reacted at an NCO:OH equivalent ratio approximately equal to two to make an NCO-terminated prepolymer. The bioactive component is dissolved in water (the chain extender) and added to the prepolymer to make a polyurethane foam.

The biodegradable, biocompatible and bioactive polyurethanes of the present invention are well suited for use as tissue engineering scaffolds. For example, the polyurethane can be coated with human osteoblastic precursor cells, which are then cultured under conditions suitable to promote cell growth. In one embodiment, the polyurethane scaffold is seeded with human osteoblastic precursor cells and the cells cultured in vitro prior to implantation in the body. In another embodiment, the polyurethane is formed in vivo and optionally seeded with cells. The isocyanate-terminated prepolymer of the present invention can, for example, be initially formed ex vivo and sterilized (for example, by autoclaving). The mixture of bioactive agents is then dissolved in sterile water. The prepolymer and water are then mixed in vivo to form a bioactive polyurethane foam which can act as a bone tissue scaffold.

Polyurethane Foams Containing Ascorbic-Acid

The use of a biodegradable, biocompatible and bioactive polyurethane of the present invention as a bone scaffold was demonstrated using an ascorbic acid-containing polyurethane-urea. The bone scaffold was synthesized using the ethyl ester of lysine diisocyanate (LDI), glycerol, polyethylene glycol (200 MW, PEG), and ascorbic acid (AA). The reaction of LDI, PEG and glycerol in a ratio of 4:1:2 (—NCO/—OH=1.05) for 5 days resulted in the formation of a viscous isocyanate-functional pre-polymer. FT-IR demonstrated the formation of urethane linkages (peak at 1725 cm⁻¹). Relatively low molecular weight polyethylene glycol (PEG; average Mn ca. 200) was used as both one of the raw materials and as a solvent. As such, no additional organic solvent was needed for any of the polymer synthesis or fabrication steps. There was thus no excess organic solvent in the reaction system. The addition of PEG to the polymer created a softer material with higher porosity and surface area.

The addition of water to the polymer viscous solution resulted in the formation of polymer foam. In that regard, water reacted with remaining isocyanate groups, forming carbon dioxide and an amine. The amine reacted further with an isocyanate group to create a urea linkage. Hence the addition of water initiated foaming and also strengthened the material via urea formation. Scanning micrographs of the polymer showed total porosity values ranging from approximately 60% to approximately 90%. Changing the amounts of water varied the porosity in the polymer foams. For example, the addition of 1 ml water into 10 gram of polymer solution produced a polymer foam with 65% porosity, while a foam of 90% porosity was obtained by adding 1.5 ml water into 10 gram of polymer solution. A cross-sectional view showed sponge-like cavities formed as a result of the liberation of CO₂ during foaming process. The pore sizes were typically distributed in the range of approximately 100 to approximately 500 μm. The cross-sectional view of the polymer showed that not only did the pores in the polymer provide a large surface area to support cell growth, but also that the pores were interconnected to allow free fluid flow for circulation of nutrients and other metabolites.

Ascorbic acid is among the most unstable vitamins, and its stability is affected by temperature, pH, salt concentration, sugar concentration, oxygen concentration, metal catalysts and enzymes. When ascorbic acid is oxidized, it changes to a yellow color. The LDI-glycerol-PEG-AA polymer foam of the present invention was heated at 100° C. for 3 hrs. After such heating a yellow color distributed homogeneously in the LDI-glycerol-PEG-AA foam. On the other hand, no yellow color was observed in an LDI-glycerol-PEG polymer heated in the same manner. This result indicated that ascorbic acid has bound to the LDI-based polymer scaffold and distributed homogeneously. It is known that sodium ascorbyl phosphate is more stable than ascorbic acid, because of hydroxy group of ascorbic acid reacted with phosphic acid. Similar, the stability of ascorbic acid in the LDI-glycerol-PEG-AA polymer of the present invention was increased by the polymerization between LDI and ascorbic acid.

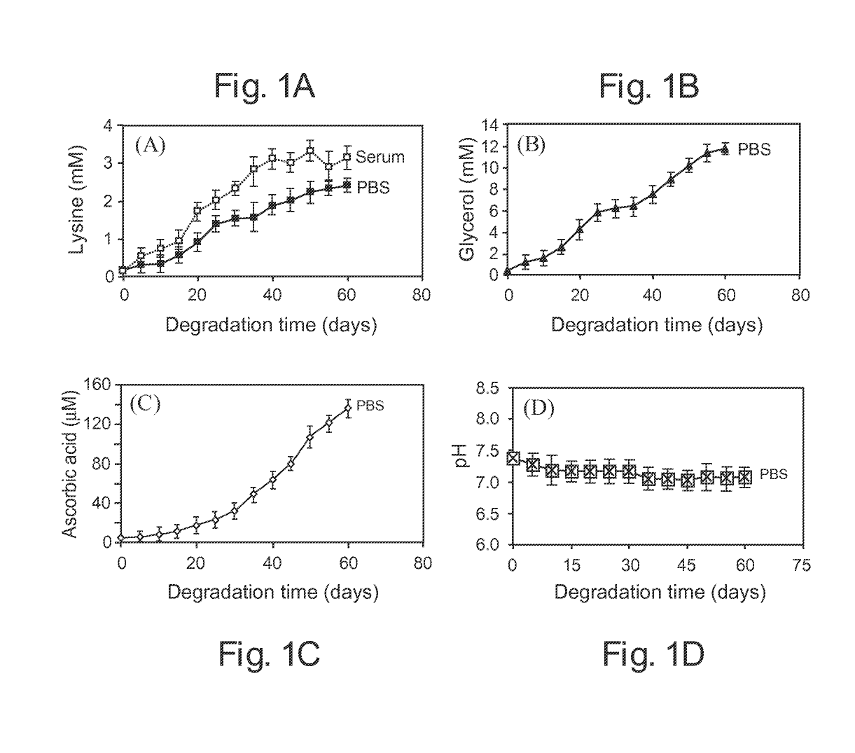

Degradation tests indicated that the LDI-glycerol-PEG-AA scaffold of the present invention was degradable, and that observable degradation products included lysine, glycerol, PEG, ascorbic acid and ethanol. Further examination of the degradation profile of LDI-glycerol-PEG-AA scaffold showed the presence of glycerol at approximately half the concentrations of lysine (see FIGS. 1A and 1B). The release of ascorbic acid was found to occur at a rate similar to that of lysine (see FIG. 1C). The results agree with the expected hydrolysis of the urethane and urea bonds of the LDI-glycerol-PEG-AA polymer, which would ultimately result in the liberation of lysine, glycerol, PEG, ascorbic acid, ethanol and CO₂. The LDI-glycerol-PEG-AA polymer was found to degrade twice as fast in a serum-containing PBS system than in a PBS only system (see FIG. 1A). This result may be explained by the enzymes contained in serum which hydrolyze peptides and amino acids easily. The degradation rate was controllable by regulating the ratio of LDI/glycerol/PEG. In general, ester linkages biodegrade more quickly that urethane or urea linkages.

The degradation products of certain biodegradable polymers (for example, those made by poly-lactide and poly-glycolide) create an acidic environment in vivo which can be detrimental to the surrounding biological system. To study whether the breakdown products of LDI-glycerol-PEG-AA scaffold of the present invention change the pH of the surrounding solution, the pH of the PBS containing 100 mg/ml polymers was measured over a period of 60 days. As shown in FIG. 1D, the degradation products of LDI-glycerol-PEG-AA polymer did not affect the pH of polymer degradation solution significantly at physiological temperature tested (that is, 37° C.).

The stability of the LDI-glycerol-PEG-AA polymer scaffold of the present invention was also tested at several conditions. The LDI-glycerol-PEG-AA polymer scaffold was stable for at least 20 months at room temperature in a tightly closed container. The container was stored in a dry place at a maximum temperature of 25° C., and the polymer was protected from light exposure. Upon exposure to light, moisture, and heat the polymer gradually darkened, but it was not determined if such darkening was correlated with any instability of the polymer scaffold.

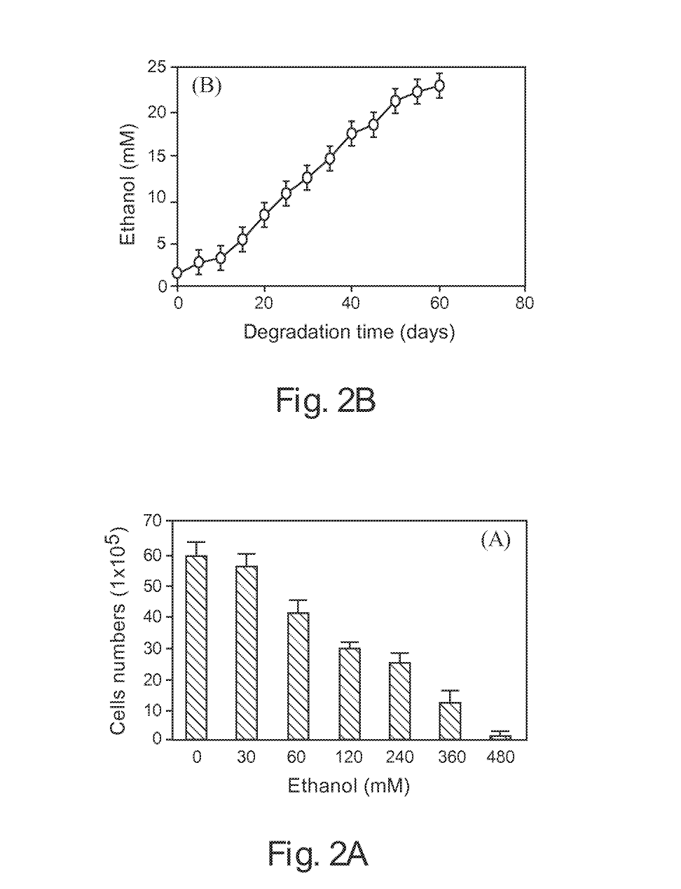

Ethanol was monitored as one of the degradation products of LDI from the ascorbic acid-containing polyurethanes of the present invention via gas chromatography. Gas chromatography showed that ethanol had a peak at retention time of 6.81 min with a concentration-dependent manner. To determine whether alcohol directly affected osteoblast function, the effect of alcohol on the cell culture was studied. The ethanol concentration as the polymer degraded was also studied. In vitro studies indicated that alcohol affected cell proliferation in a dose-dependent manner. However, if the alcohol concentration was lower than 30 mM (0.5%, v/v), there was no apparent harmful effect on the cells. Gas chromatography results suggested that ethanol was slowly liberated from the polymer and that the highest concentration of ethanol in the polymer degradation products was 24.2 mM, which was lower than the harmful concentration determined in the cell culture studies (see FIGS. 2A and 2B).

Green fluorescent protein-transgenic mouse bone marrow cells (GFP-MBMC) culture results indicated that the composition of the LDI-glycerol-PEG-AA scaffold of the present invention had a significant effect on cellular proliferation. Fluorescent micrographs of the cells on the LDI-glycerol-PEG-AA scaffold for various time intervals showed that following seeding, the cells spread on the polymer surface, and gradually adhered to the polymer within few hours. Continuous culture of GFP-MBMC on the scaffold for 7 days showed that GFP-MBMCs retained their morphology, similar to those grown on the tissue culture plate.

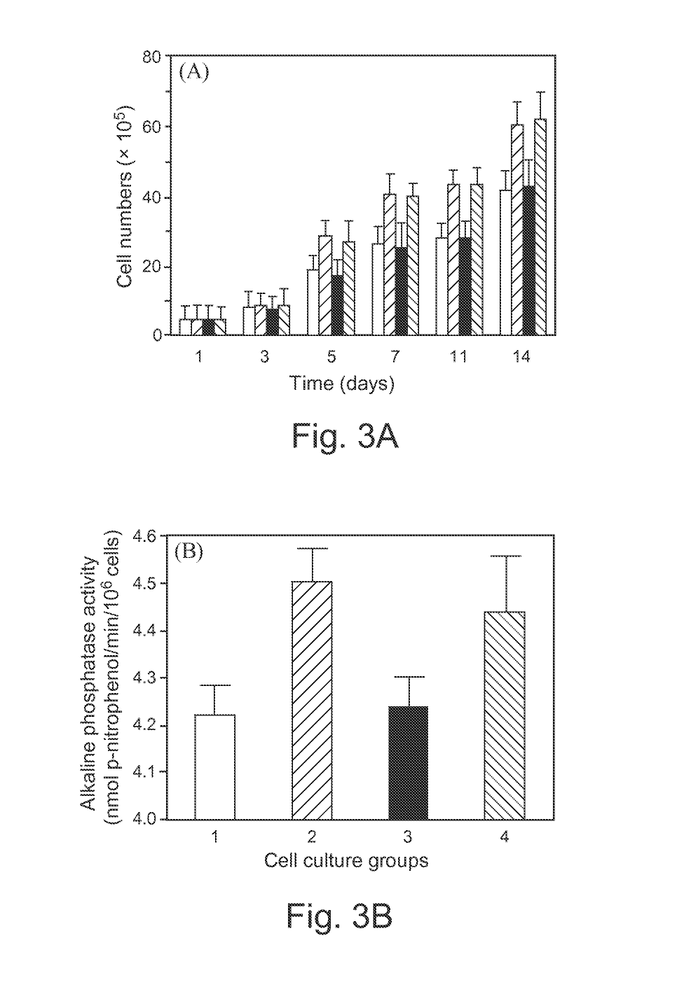

The LDI-glycerol-PEG-AA scaffold of the present invention promoted GFP-MBMC proliferation (see FIG. 3A). The cell proliferation of GFP-MBMC grown on the LDI-glycerol-AA scaffold was increased significantly during days 4 to 14. A similar increase relative to the control was seen when GFP-MBMC was cultured in the medium with 30 μg/ml ascorbic acid during days 4 to 14 (see FIG. 3A).

Alkaline phosphatase, an early marker of osteoblasts, is frequently used to assess the osteoblastic character of isolated cells. Tissue culture results of the present studies showed that the LDI-glycerol-PEG-AA scaffold stimulated the secretion of alkaline phosphatase and type I collagen of mouse bone marrow cells. This stimulatory effect was similar to that observed after the addition of ascorbic acid directly into culture medium. Alkaline phosphatase activity increased more for cells grown on a scaffold made from the LDI-glycerol-PEG-AA polymer of the present invention than that on a scaffold made from LDI-glycerol-PEG only. A similar result was seen in the cells grown in media with and without ascorbic acid (see FIG. 3B).

Collagen type I is the major organic macromolecule in bone matrix, and is primarily synthesized as a large procollagen molecule containing additional propeptides at both ends of its three-polypeptide chains. The expression of the osteoblast phenotype is regulated by a series of factors, including growth factors, glucocorticoids, parathyroid hormone, and 1,25-dihydroxyvitamin D₃; however, differentiation and mineralization seem to require the presence of an extra cellular collagen matrix. Ascorbic acid has been shown to be necessary both for the production of the collagen matrix and for the expression of osteoblast markers, such as alkaline phosphatase and osteocalcin. Alkaline phosphatase is an osteoblastic enzyme related to bone mineralization and differentiation. Histochemical staining showed that in vitro re-mineralization results followed trends similar to those found for alkaline phosphatase activity assay as a function of culture condition. The results further indicated that the ascorbic acid-containing polyurethane scaffold of the present invention enhanced type I collagen synthesis in mouse bone marrow cells.

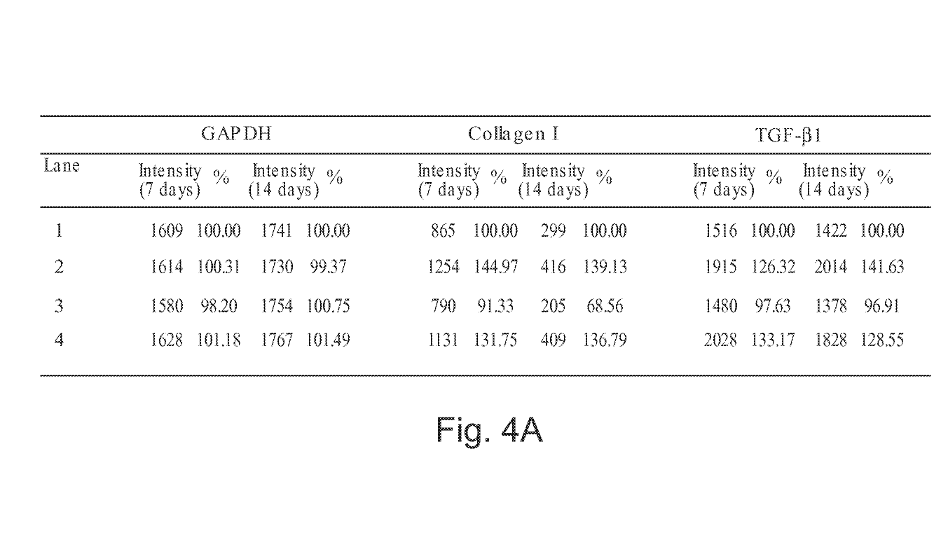

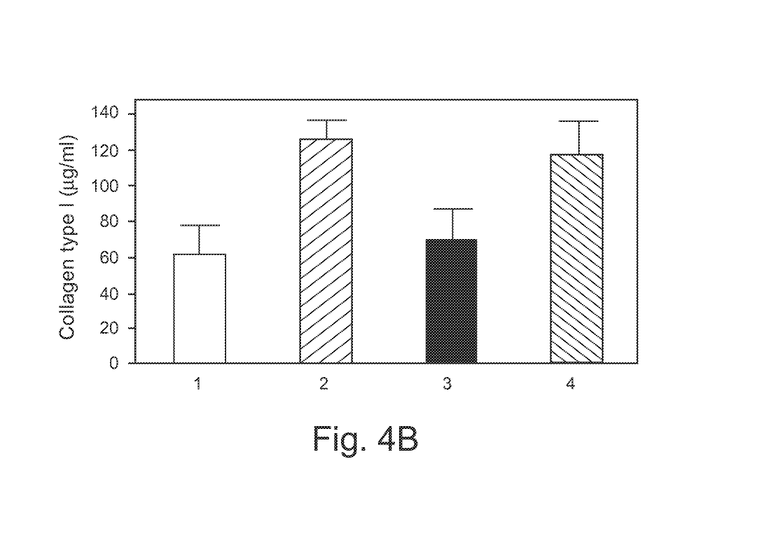

In that regard, the ability of GFP-MBMC to express mRNA for collagen type I following culture under the four culture conditions was studied. RT-PCR showed that the cells grown on the scaffold made from LDI-glycerol-PEG-AA showed a significant increase of mRNA for collagen type I than that on the scaffold made from LDI-glycerol-PEG only (P<0.005, see FIG. 4A). Cells grown in the media exhibited a lower level of collagen type I mRNA relative to culture in media containing ascorbic acid.

The concentration of collagen type I determined in the cells grown in the scaffold made from LDI-glycerol-PEG-AA was higher than that of the cells grown in the scaffold made from LDI-glycerol-PEG (see FIG. 4B). The concentration of collagen type I in the cells grown in the medium with ascorbic acid and on the LDI-glycerol-PEG-AA scaffold was two times higher than that of the cells grown on the LDI-glycerol-PEG scaffold or tissue culture plate after 7 days culture (P<0.005). This result indicated that the ascorbic acid-containing scaffold stimulated bone cells to synthesize and secrete collagens.

Western blotting test also showed that the higher concentrations of type I collagen were secreted by GFP-MBMC grown in the medium with ascorbic acid and grown on the LDI-glycerol-PEG-AA scaffold.

Polyurethane Foams Containing Dexamethasone and β-Glycerophosphate

Bioactive polymers containing covalently bound dexamethasone and β-glycerophosphate were also synthesized and studied for potential use as bone scaffolds. In several studies, mouse bone cells (OPC) (9.6×10⁴/well) were cultured in a 6-well tissue culture plate without polymer foam (blank column) with Dulbecoo's modified Eagle's medium only (DMEM); with 5 mM β-glycerophosphate (β-GP); with 100 nM dexamethasone (Dex); 50 μM ascorbic acid (Vc); with 50 μM ascorbic acid and 5 mM β-glycerophosphate (Vc+β-GP); with 50 μM ascorbic acid and 100 nM dexamethasone (Vc+Dex); with 50 μM ascorbic acid, 100 nM dexamethasone and 5 mM β-glycerophosphate (Vc+Dex+β-GP) for 4 weeks.

The same cells were cultured in a 6-well plate with the LDI-PEG400-glucose polymer (red column) using DMEM only. The polymer foams were synthesized with lysine ethyl ester diisocyanate (LDI), glucose and PEG only (DMEM); with LDI, glucose, PEG and β-glycerophosphate (β-GP); with LDI, glucose, PEG and dexamethasone (Dex); with LDI, glucose, PEG and ascorbic acid (Vc); with LDI, glucose, PEG, ascorbic acid and β-glycerophosphate (Vc+β-GP); with LDI, glucose, PEG, ascorbic acid and dexamethasone (Vc+Dex); and with LDI, glucose, PEG, ascorbic acid, 3-glycerophosphate and dexamethasone (Vc+Dex+β-GP). The polymer foam was cut into small disks with a 1 mm thickness and a 30 mm diameter (100 mg/disk/well). OPC (9.6×10⁴/disk) was cultured with DMEM for 4 weeks. The alkaline phosphatase activity was determined by the OD at 405 nm in the medium with or without polymer.

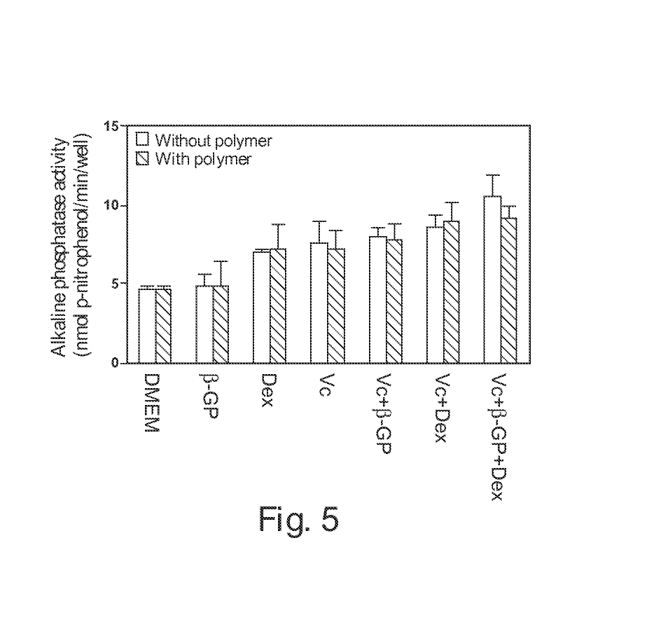

The results as illustrated in FIG. 5 indicated that the alkaline phosphatase activity of OPC was significantly increased by dexamethasone and ascorbic acid, while β-glycerophosphate promoted the alkaline phosphatase activity of OPC more moderately.

In another study, OPC (9.6×10⁴/well) was cultured in a 6-well tissue culture plate with Dulbecoo's modified Eagle's medium only (DMEM); with 100 nM dexamethasone (Dex); in the polymer foam synthesized by LDI, glucose, PEG 400 and cultured with DMEM only (Foam); and with the polymer foam synthesized by LDI, glucose and PEG and dexamethasone (Foam-Dex).

The polymer foam was cut into small disks with 1 mm thick and 30 mm diameter (100 mg/disk/well). OPC (9.6×10⁴/disk) was cultured with DMEM for two weeks. The cell proliferation was determined by the MTT method.

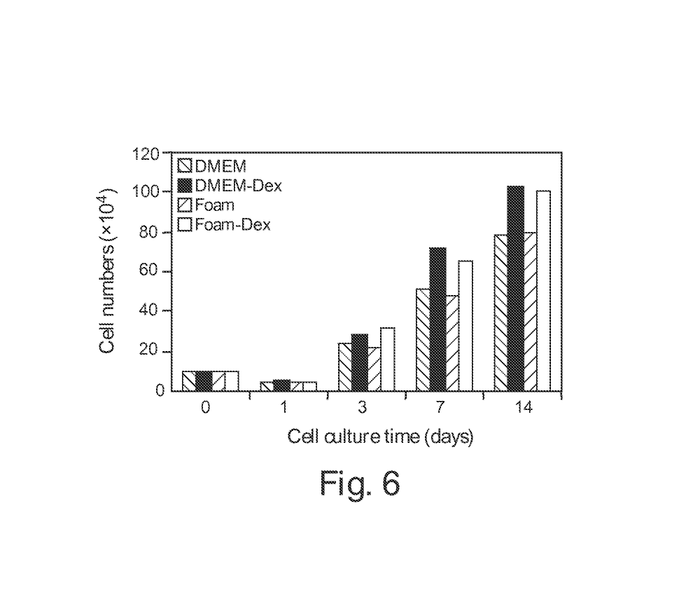

The results as illustrated in FIG. 6 indicated that the proliferation of OPC grown in the dexamethasone-containing polymer foam was increased significantly during 4 to 14 days. A similar increase relative to the control was seen days 4 to 14 when OPC was cultured in the medium with dexamethasone (100 nM).

Polyurethane Foams with Nucleic Acid/Virus Vector; Gene Delivery (RUN2x)

Runx2 is a central regulator of osteoblast differentiation and function and a transcription factor, which binds to the osteoblast-specific cis-acting element 2 (OSE2) present in the promoter of the osteocalcin gene. Several studies have established that Runx2 is required for in vivo bone formation as well as for maturation of hypertrophic chondrocytes and for osteoblast differentiation. Forced expression of Runx2 in nonosteoblastic cells has been found to induce expression of osteoblast-specific genes, and the effects of Runx2 overexpression on in vitro matrix mineralization have been determined. Such studies suggested that Runx2 gene delivery is a tool for enhanced bone generation by encouraging specific cellular osteogenic responses.

In general, gene therapy is a promising approach for treatment of inherited or acquired diseases. An obstacle to the successful clinical application of gene therapy, however, is the development of effective gene transfer carriers. Such carriers must not be pathogenic or toxic to patients (that is, they must be biocompatible). Administration of DNA alone has yielded successful gene transfer for a number of isolated applications, but with a limited spectrum of organ-specific expression. In that regard, naked DNA is highly sensitive to serum nuclease digestion. Furthermore, naked DNA and DNA plasmid, which is administered to particular physiological locations, often escapes from the target sites and diffuses to tissues and organ systems distant from its original placement.

In another study of the present invention, a polymer-Runx2 complex for gene delivery was synthesized. A lysine diisocyanate-based, PEG-containing polyurethane of the present invention was found to sustain Runx2 plasmid stability, localization and subsequent transfection in vitro. An injectable polyurethane was synthesized using LDI, PEG and O,O′-Bis(2-aminopropyl)-polypropylene glycol 300 (APPG). Runx2-pIRESneo plasmid was combined with the LDI-PEG-APPG polyurethane polymer. The polyurethane-Runx2 scaffold was then used for the transfection of NIH 3T3 cells. The transfection effect of Runx2-pIRESneo plasmid was measured with and without LDI-PEG-APPG polymer by means of the mRNA expressions of Runx2, osteocalcin (OCN), alkaline phosphatase (ALP), and collagen pro-α-I type I (Collagen I) in NIH 3T3 cells after three weeks. The results indicated that polyurethanes of the present invention can act as a carrier for gene delivery.

In general, injectable forms of the polyurethanes of the present invention preferably foam substantially to completion in no more than 15 minutes. More preferably, the injectable polyurethane foams substantially to completion in no more than 10 minutes. Most preferably, the injectable polyurethane foams substantially to completion in no more than 5 minutes. Control of the time required for foaming can be achieved through the differences in the reaction rates of amine groups and hydroxyl groups with isocyanate groups. In general, amine groups, and particularly, primary aliphatic amine groups, react with isocyanate groups significantly more quickly than do hydroxyl groups. One can, for example, form an isocyanate terminated prepolymer of the present invention as described above and subsequently react the prepolymer with a polyfunctional amine chain extender and a bioactive agent. The prepolymer and the chain extender/bioactive agent can, for example, be injected separately to contact in vivo and form the foam in vivo. Alternatively a one step or single shot synthetic route as described above can be use in which, for example, the amine-polyfunctional chain extender and the bioactive agent are injected from one syringe and the multifunctional isocyanate are injected from another syringe to contact in vivo and form the foam in vivo.

The LDI-based injectable polyurethane scaffold of the present invention was synthesized using the ethyl ester of LDI, PEG and APPG by a one-step injection reaction. The injection of LDI into a mixture of PEG and APPG with pIRESneo-Runx2 plasmid resulted in the formation of polymer foam. A scanning micrograph of the polymer showed that sponge-like cavities apparently formed as a result of the liberation of CO₂ during foaming process. The porous structure was a three-dimensional continuous fibrous network. The porosity of the polymer varied in various areas, with pore sizes in the range of approximately 50 to approximately 250 μm in diameter. The cross-sectional view of the polymer showed that the pores provided a large surface area to support cell growth, and the pores were interconnected, thereby facilitating free fluid flow for circulation of nutrients and other metabolites.

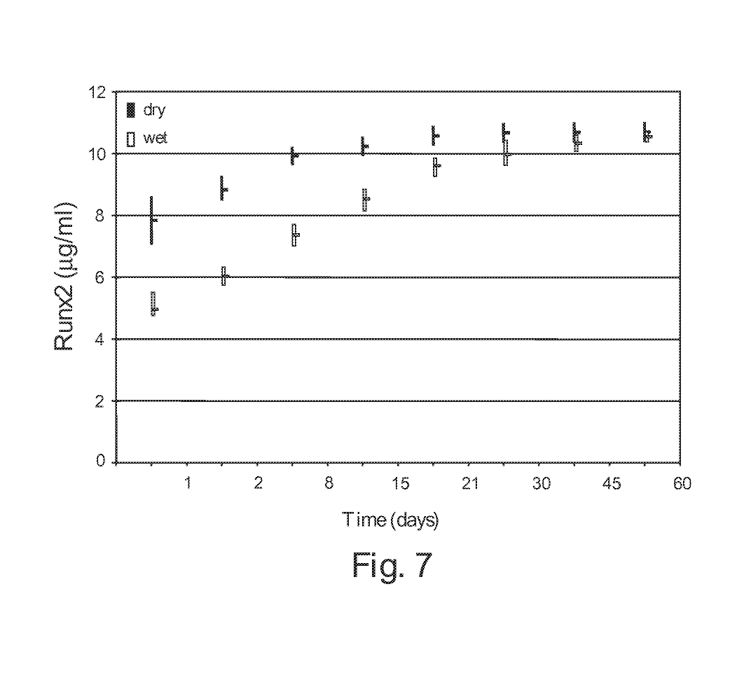

“Wet” and “dry” LDI-PEG-APPG-pIRESneo-Runx2 matrices (10 μg plasmid/piece polymer; 100 mg polymer/piece) were immersed in phosphate-buffered saline (PBS) and incubated under physiological conditions for 60 days. Release kinetics of pIRESneo-Runx2 plasmid from carrier matrices was determined daily by spectrometry. In general, and as further described below, in the wet scaffold, pIRESneo-Runx2 plasmid was incorporated into the polymer during polymerization, whereas in the dry scaffold, the polymer matrix was first formed and allowed to dry before addition of the pIRESneo-Runx2 plasmid to the matrix. As shown in FIG. 7, the Runx2-pIRESneo plasmid released faster from the dry scaffold than from the wet scaffold. By the end of day 1, about 80% of the plasmid was released from the dry scaffold, however, only 45% of the plasmid was released from the wet scaffold. By day 15, 100% of pIRESneo-Runx2 was released from the dry scaffold, but 17% of the plasmid still remained in the wet scaffold. By the end of the 60 days, all pIRESneo-Runx2 plasmid released from both kinds of matrices.

The results indicated that the pIRESneo-Runx2 plasmid contained in the scaffold is released from the polymer. The rate at which Runx2 plasmid is released can be modified by altering the conditions of matrix synthesis. There are two different mechanisms of the DNA plasmid released from the wet and the dry scaffolds. In wet polymers, the Runx2 plasmid is bonded to LDI-PEG-APPG scaffold generally homogeneously. The plasmid release accompanies the degradation of the polymer and is therefore gradual. In the dry polymers, Runx2 plasmid is added heterogeneously after scaffold formation. A large proportion of Runx2 plasmid in the dry polymer may be attached to the surface of the scaffold; therefore, initial rapid release was relatively independent of the polymer degradation and was fast during the first 24 hours. After 24 hours, however, release rates appeared to be influenced by the degradation of the polymer scaffolds. Loading the Runx2 plasmid on the dry polymer scaffold by the conventional swelling/absorption mechanism may introduce steric hindrances resulting in heterogeneous DNA plasmid loading distribution and release. The erratic incorporation of the plasmid utilizing a dry polymer triggered unreproducible release of the plasmid. A generally homogeneous DNA plasmid loading and distribution was obtained by using a wet scaffold procedure. Moreover, the DNA plasmid itself may covalently link to the polymer matrix during the wet polymer scaffold cross-linking process. The consistent pattern of plasmid release from the wet matrix preparation are advantageous for evaluating gene delivery, and the wet matrix preparation was used for the subsequent experiments discussed below.

X-gal staining results indicated that naked LacZ-plasmid was not transfected into NIH3T3 cells. About 13.43% of the transfection efficiency was found in NIH3T3 cells by means of calcium phosphate precipitation technique. However, 36.64% of the transfection efficiency was found in NIH3T3 cells transfected with LacZ plasmid by LDI-PEG-APPG-lacZ polymer.

The transfection efficiency of the polyurethane scaffold was investigated over three weeks. At three weeks following pIRESneo-Runx2 plasmid transfection, enhanced Runx2 gene expression was found in NIH3T3 cells transfected pIRESneo-Runx2 plasmid by LDI-PEG-APPG polymer. Compared with the transfections using the polymer, a less apparent Runx2 gene expression was found in the cells transfected with pIRESneo-Runx2 plasmid without polymer. The expression of GAPDH, a housekeeping gene, indicated equal amounts of total mRNA were used in RT-PCR experiment.

The osteocalcin expression on the same samples was investigated by RT-PCR. A faint expression of osteocalcin was detected in NIH3T3 cells transfected with pIRESneo-Runx2 plasmid and incubated for three weeks without polymer. However, a stronger osteocalcin expression was found in NIH3T3 cells cultured for three weeks on the polymer containing pIRESneo-Runx2 compared with controls.

After three weeks transfection, a stronger expression of type I procollagen alpha I was found in the cells cultured on polymer containing pIRESneo-Runx2 compared with controls. A biochemical assessment of alkaline phosphatase activity on the cells was studied at days 21 using a Sigma kit. The result showed that NIH3T3 cells transfected pIRESneo-Runx2 plasmid by LDI-PEG-APPG polymer had a higher alkaline phosphatase activity than that of the cells transfected pIRESneo-Runx2 plasmid without polymer.

The above results indicated that an injectable, rapid-foaming polymer was successfully developed for gene delivery. pIRESneo-Runx2 plasmid recombined with LDI-PEG-APPG polymer to constitute gene carrier matrix. The Runx2-containing polyurethane scaffold of the present invention had a stimulant effect on the secretion of alkaline phosphatase and type I collagen from NIH3T3 cells. Higher transfection efficiency was found in LDI-based polymer-plasmid system. Runx2 plasmid was transfected into NIH3T3 cells by LDI-based urethane polymers of the present invention and induced the differentiation of the fibroblast into an osteoblast like phenotype. NIH3T3 cells transfected with Runx2 plasmid transfection by LDI-based scaffold includes Runx2 gene, osteocalcin, alkaline phosphatase, and collagen type I. In the studies of the present invention, the expression of collagen type I in non-transfected controls was not detected. The results indicate that LDI-Runx2 system of the present invention may be a useful tool for gene delivery, cell transfection and ultimately, bone regeneration.

Segmented Biodegradable Polyurethanes-Load-Bearing Applications

Biocompatible and biodegradable polyurethanes of the present invention can be synthesized with mechanical properties suitable to be useful for load-bearing tissue engineering applications, including, for example, cellular elastomers for the knee-joint meniscus, semi-rigid foams for spinal fusions, and thermoplastic elastomers for cardiovascular tissue. It is not necessary to make polyurethanes having the same properties as conventional materials, but rather to prepare materials with a broad range of structures to enable preparation of scaffolds having mechanical properties matching those of the tissue for which they are designed. The mechanical properties of the load-bearing and other polyurethanes of the present invention are governed by known structure/property relationships. In general, if the structures of the biocompatible intermediates of the present invention are comparable to those of conventional intermediates, the resulting biocompatible polyurethanes of the present invention will have mechanical properties similar to those of conventional polyurethanes. The hardness of polyurethanes generally increases with increasing melting temperature of the hard segment. Also, the compression modulus increases with increasing hardness. The melting temperature of hard segment preferably varies between approximately 50 and 300° C., more preferably between 100 and 250° C., and most preferably between 100 and 200° C.

If only natural metabolites are used as raw materials, the load-bearing polyurethanes of the present invention degrade by hydrolysis of ester, urethane, and urea groups to resorbable biomolecular components. Provided the concentrations of the degradation products are not excessively high compared to normal physiologic conditions, the polyurethanes of the present invention are both biodegradable and biocompatible.

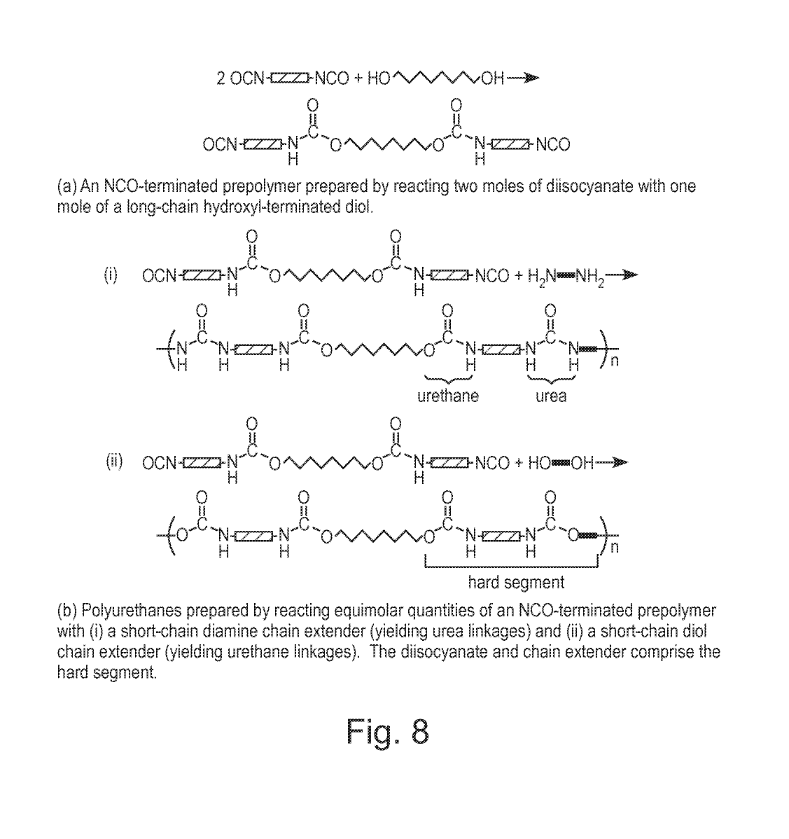

As described above, polyurethanes are often prepared via a two-step process, as shown in FIG. 8. In the first step, an NCO-terminated prepolymer is typically prepared by reacting two moles of diisocyanate with one mole of a hydroxyl-terminated polyether or polyester. In the second step, the polyurethane is typically prepared by reacting one mole of prepolymer with one mole of either a diamine or diol chain extender. The diisocyanate and chain extender form the hard segment and the polyether (or polyester) polyol forms the soft segment. Polyols that result in amorphous, noncrystalline soft segments are preferred in the segmented polyurethanes of the present invention. The hard segments (which are derived from the reaction of a diisocyanate and a chain extender) and soft segments are connected by urethane or urea linkages. Isocyanates react with diols to form urethane linkages and with diamines to form urea linkages.

Because of differences in polarity, the hard and soft segments phase-separate to form a two-phase morphology. The extent of phase-separation increases as the difference in polarity between the hard and soft segment increases. The urea and urethane linkages in adjacent chains are capable of hydrogen bonding, resulting in inter-chain attractive forces and aggregation of hard segments. Under certain conditions, the phase-separated hard segments form paracrystalline domains, which have a significant hardening effect on properties. The inter-chain hydrogen bonds act as physical cross-links which, unlike chemical cross-links, can be disrupted at elevated temperatures or in solvents. The hydrogen bonds between urea groups are stronger than those between urethane groups. Therefore, amine-extended polyurethanes typically have higher melting points than diol-extended materials. Unbranched, symmetric diamine chain extenders often yield polyurethanes with melting points above the decomposition temperature (˜250° C.), rendering them non-thermoplastic.

A number of structure-property relationships for polyurethanes are summarized in Table 1 below. Symmetric diisocyanates and chain extenders with an even number of carbon atoms pack into a crystal lattice more effectively than asymmetric molecules with an odd number of carbon atoms. The stiffness of the hard segment also affects mechanical properties. Aromatic groups in the backbone introduce large, flat, rigid units that significantly increase the stiffness of the backbone and enhance crystallinity. The i-electron interactions between adjacent aromatic rings of symmetric aromatic diisocyanates also result in interchain attraction. Because side branches hinder molecules from packing into a crystal lattice, linear molecules promote hard segment crystallinity more than branched molecules. Short side groups (e.g., CH₃) hinder packing of the chain into the lattice, while longer side chains may form small crystallites, which often result in more waxy properties. Polyurethane elastomers are typically linear molecules with only a small degree, if any, of chemical cross-linking

TABLE 1

| Increase Hard | Decrease Hard |
|---|---|
| Segment Crystallinity | Segment Crystallinity |
| High concentration of urea and | Low concentration of urea and |
| urethane linkages in the hard | urethane linkages in the hard |
| segment | segment |
| Linear backbone | Branched backbone |
| Symmetric hard segment | Asymmetric hard segment |
| Even number of carbon atoms in the | Odd number of carbon atoms in the |
| chain extender backbone | chain extender backbone |
| Phenyl groups in the backbone | No phenyl groups in the backbone |

In general, hardness and modulus increase with increasing hard segment content, which can be controlled by varying the relative proportions of chain extender and polyol. Hardness and modulus also increase with increasing phase-separation and crystallinity of the hard domains, which are affected not only by the structure of the backbone and interchain attraction, but also by the thermal and mechanical history of the material. For example, annealing between the glass transition and melting temperatures can increase the percent crystallinity. Preferably the percent crystallinity of the hard segments is at least 5%. More preferably, the percent crystallinity of the hard segments is at least 20%.

In the segmented polyurethanes of the present invention, diol and diamine chain extenders of relatively high molecular weight are preferably synthesized by coupling two moles of a biomolecule with hydroxyl and/or amine functionality with one mole of a short-chain biocompatible diisocyanate. The chain extenders can, for example, be synthesized by coupling biomolecules either through an esterification reaction or through the isocyanate reaction. The resultant macrodiols and macrodiamines advantageously enable the synthesis of symmetric chain extenders with a relatively high molecular weight. In several embodiments, the chain extenders have an even number of carbon atoms. Chain extenders of relatively high molecular weight are preferred for use in the segmented polyurethanes of the present invention because the biocompatible diisocyanates use therein are of low molecular weight. The chain extenders preferably increase the molecular weight of the hard segments to enable phase separation and crystallization of the hard segments. Preferably, the molecular weight of the chain extender of the segmented polyurethanes of the present invention is at least 100 Daltons. In several preferred embodiments, the molecular weight of the chain extender of the segmented polyurethanes of the present invention is in the range or approximately 100 to approximately 1000 Daltons. In several other preferred embodiments, the molecular weight of the chain extender of the segmented polyurethanes of the present invention is in the range or approximately 200 to approximately 750 Daltons.

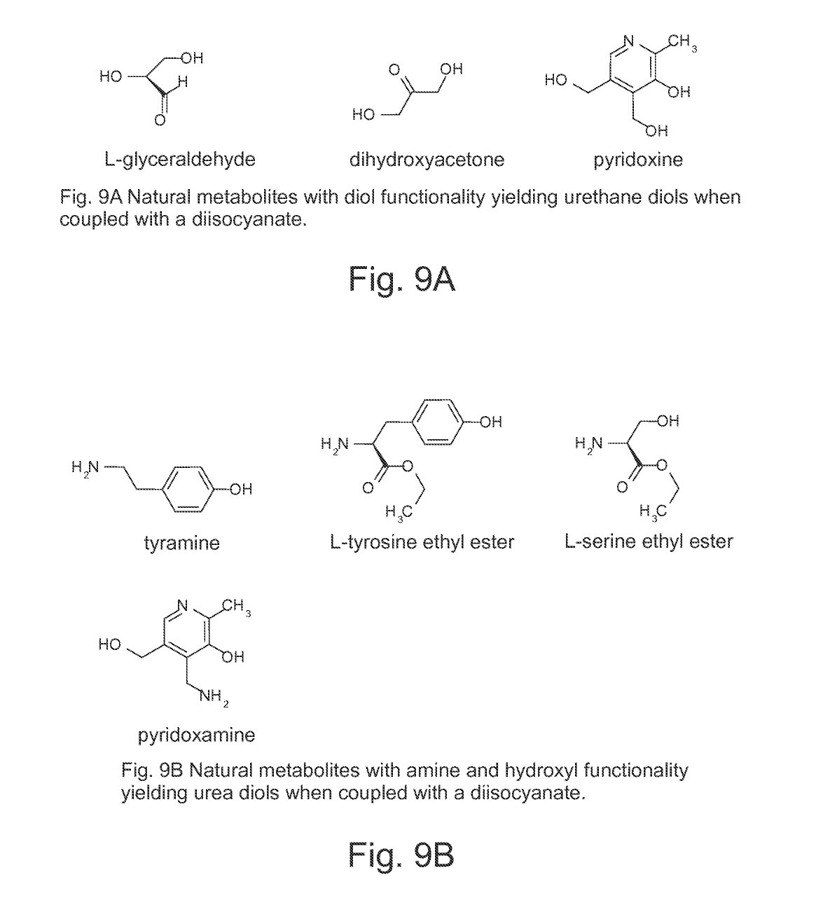

Several natural metabolites/biomolecules suitable for use in the segmented polyurethanes of the present invention and having hydroxyl and/or amine functionality are shown in FIGS. 9A and 9B. Tyrosine (Tyr) is a non-essential amino acid for human development which is a precursor for the synthesis of thyroid hormones and neurotransmitters (e.g, dopamine) Because phenol is a stronger acid, and therefore a weaker nucleophile, than aliphatic alcohols, it reacts considerably more slowly Tyramine (TyA) is a decarboxylation product of tyrosine found in mistletoes and ripe cheese. Serine (Ser) is a non-essential amino acid for human development found in the active site of serine proteases (e.g., trypsin) that is a metabolic precursor for purine synthesis via the de novo pathway. Glyceraldehyde (GlA, an aldose) and dihydroxyacetone (DHA, a ketose used as an artificial tanning agent in commercial products) are interconvertible isomers through a common enediol. Glycerose, the equilibrium mixture of the two monosaccharides, has a significant role in the fermentation of sugars. Pyridoxine and pyridoxamine are vitamins of the B₆ complex, which are found in many foods, such as yeast, cereals, and liver.

Diurethane diol chain extenders are, for example, prepared by reacting two molecules of a natural metabolite/biomolecule with primary hydroxyl functionality (see FIG. 9A) with one molecule of a biocompatible diisocyanate. Diurea diol chain extenders are prepared by reacting two molecules of a natural metabolite/biomolecule with both primary amine and hydroxyl functionality (FIG. 9B) with one molecule of a biocompatible diisocyanate in the absence of catalyst. The reaction can, for example, be conducted in a suitable solvent (e.g., DMF) with or without catalyst at 60-100° C.

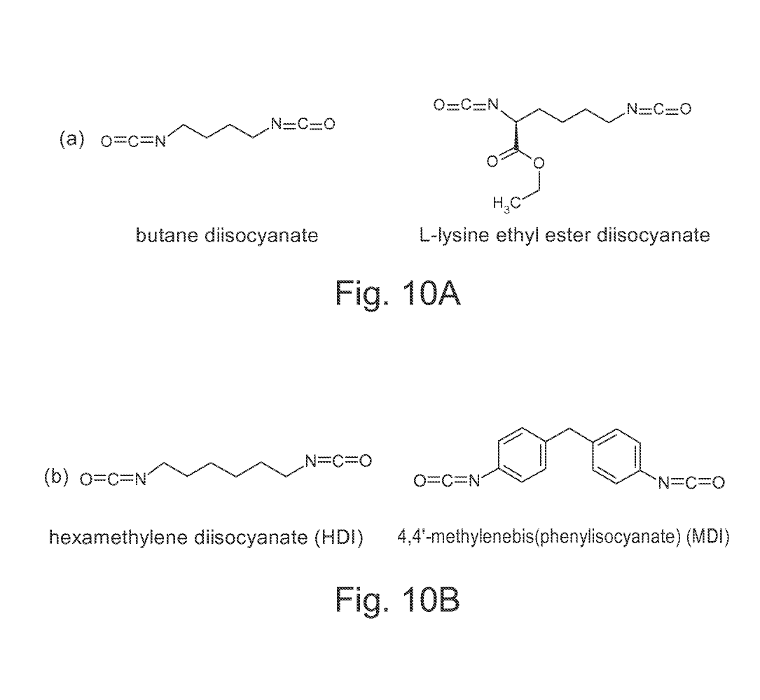

Preferably diisocyanates include butane diisocyanate (BDI), lysine diisocyanate, lysine methyl ester diisocyanate and lysine ethyl ester diisocyanate (in general, lysine diisocyanate, lysine methyl ester diisocyanate and lysine ethyl ester diisocyanate are sometimes referred to herein individually or collectively as LDI), the chemical structures of which are shown in FIG. 10A. The primary decomposition product of BDI is putrescine (1,4-butanediamine, BDA), which is a precursor of spermidine that is essential for cell division in mammals. LDI decomposes to form lysine and ethanol as described above. When synthesizing urea diol chain extenders in the absence of a catalyst, the amine groups react with the isocyanate groups to near completion while the hydroxyl conversion is low as a result of its considerably lower reactivity. Because the amine group on the amino acid react in the absence of a catalyst while the hydroxyl group do not (at least appreciably), it is possible to form urea diols with a uniform size distribution without an excess of diisocyanate.

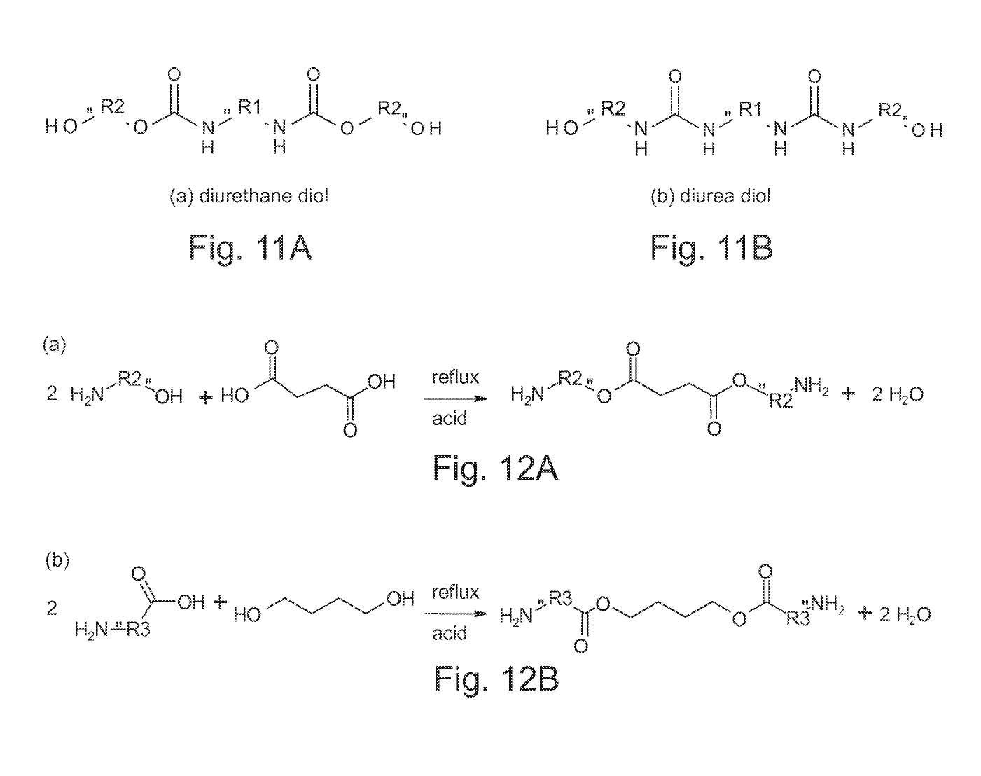

Structures of the diurethane diol and diurea diol chain extenders of the present invention are shown in FIGS. 11A and 11B, respectively. Using the synthetic routes described above it is possible to significantly vary the structure of the hard segment and thereby the mechanical properties. For example, branches and phenyl groups in the backbone, and the relative concentrations of urethane and urea linkages in the backbone can be varied to vary the mechanical properties of he polyurethane.

Diester diamine chain extenders are prepared by coupling two molecules of a natural metabolite with hydroxyl and amine functionality (see FIG. 9B) with one molecule of a biocompatible diacid. As described above, the amine can be protected. Suitable diacids, include for example, succinic acid (SucA, butanedioc acid), which is a biomolecule found in fungi and lichen, and adipic acid, which is found in beet juice. Both succinic acid and adipic acid are by-products of ω-oxidation in the endoplasmic reticulum. Two molecules with both hydroxyl and amine functionality (FIG. 9B) can, for example, be coupled with one molecule of succinic acid by the Fischer esterification reaction as shown in FIG. 12A. The reaction is performed under reflux conditions in toluene using p-toluene sulfonic acid as a catalyst and is driven to completion by removing the water. The ester groups hydrolytically degrade to succinic acid and the amino acid from which the chain extender was made.

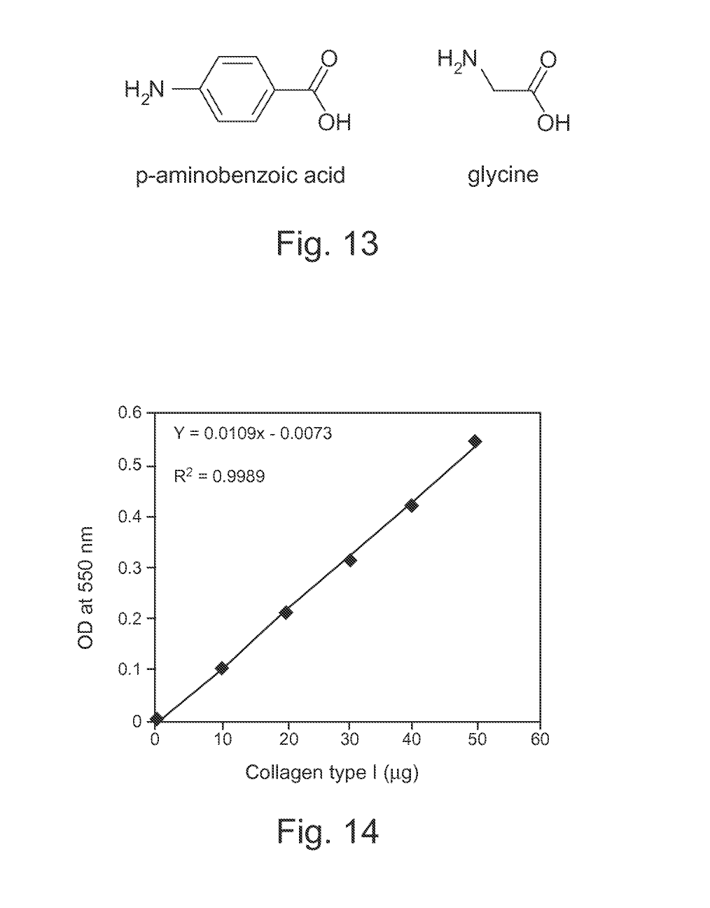

Diester diamines can also be prepared by coupling two molecules of a natural metabolite/biomolecule with carboxylic acid or ester and amine functionality (see FIG. 13) with one molecule of a biocompatible diol, as shown in FIG. 12B. U.S. Pat. No. 6,111,129 describes a process for the synthesis of diester diamines from p-aminobenzoic acid and linear alkyl diols via transesterification. Glycine (Gly) is a non-essential amino acid that acts as an inhibitory neurotransmitter. p-aminobenzoic acid (pABA) is found in many biological organisms as a vitamin B complex factor, such as Baker's yeast (5-6 ppm) and brewer's yeast (10-100 ppm). Butanediol is a suitable diol for the esterification; it is not a biological molecule, but it has been used previously to prepare biodegradable polyurethanes.

### Examples

### Example 1

### LDI-Glycerol-PEG-Ascorbic Acid Polyurethane Polymers

Materials.

All chemicals were analytical grad and from Sigma (St. Louis, Mo.) unless otherwise stated. Polyethylene glycol (average Mn ca. 200, PEG) was from Aldrich Chemical Company, Inc. (Milwaukee, Wis.). Dulbecco's Modified Eagle Media (DMEM) was from Life Technologies (Grand Island, N.Y. 14072, USA), and molecular biology reagents were from Perkin Elmer (Norwalk, Conn.).

### Example 1a

### Synthesis of LDI-Glycerol-PEG-AA Polymer

Lysine diisocyanate ethyl ester (LDI) was synthesized according the method described by Zhang et al. See Zhang, J. Y., Beckman, E. J., Piesco, N. P., and Agarwal, S. A new peptide-based urethane polymer: synthesis, biodegradation, and potential to support cell growth in vitro. Biomaterials 21, 1247-1258, 2000. The ascorbic acid containing polymer scaffold (LDI-glycerol-PEG-AA) was synthesized as follows: 35 mg ascorbic acid, 1.6 g PEG 200 (8 mmol, —OH 16 mmol) and 1.6 g glycerol (17.39 mmol, —OH 52.17 mmol) were mixed in a dry round-bottom flask, which was then flushed with nitrogen and fitted with a rubber septum. Subsequently, 7 ml of LDI (35.84 mmol, —NCO 71.67 mmol) were added to the flask with a syringe. The reaction mixture was stirred in the dark at room temperature for 5 days. The formation of urethane linkages was monitored by FT-IR spectra. When FT-IR spectra (specifically the peak at 2165 cm⁻¹) showed that approximately 90% of the initially present —NCO group had reacted to form urethane linkages, water (100 μl/g pre-polymer) was added, and the mixture was stirred for 30 min to generate a polyurethane-urea foam. The ascorbic acid concentration in this polymer foam was 3.09 mg ascorbic acid/g polymer.

### Example 1b

### Measurement of Ascorbic Acid Distribution

To test the distribution of ascorbic acid in the LDI-glycerol-PEG-AA polymer foam, three random pieces of the polymer foam were cut and heated at 100° C. for 3 hrs. Ascorbic acid distribution was measured by the appearance of yellow color of the LDI-glycerol-PEG-AA foam, and the LDI-glycerol-PEG polymer foam was used as a control and treated the same as the LDI-glycerol-Peg-AA polymer foam.

### Example 1c

### Pore Sizes of the Polymer Foam Assay

Visualization of the polymer foam was performed by scanning electron microscopy (SEM). Three random pieces from each polymer foam were selected from different areas, mounted on SEM sample stubs, and coated with gold/palladium and examined under a JOEL scanning microscope with an accelerating voltage of 5 kV. The pore size distribution of the polymer foam was analyzed by using the public domain NIH Image program available at http://rsb.info.nih.gov/nih-image.

An example of this image analysis procedure is described herein to provide a better understanding of the meaning of pore size calculated by this method. First, an area of the micrograph was selected for image analysis. Brightness and contrast of each SEM photograph were carefully adjusted to the same level, because the pore size measurement by image analysis software was based upon the gray-scale of the image. Thresholding (selecting reasonable pore area based on gray value) was then performed. Thresholding should be carefully done, because the pore diameter measured can be seriously affected by thresholding values. Therefore, the same value of thresholding was applied to all image analyzed in this study. The validity of the thresholding level was confirmed by comparing the image before and after thresholding, particularly comparing the position and shape of pores in the original image with their corresponding ones in the thresholded image. If a mismatch was found between the original and thresholded images, thresholding would be performed again until there was an exact match in shape, size, and location of the corresponding pores. After calibrating with a known scale, each pore was measured and labeled to decide the validity of the measurement. The diameter of a pore was obtained by averaging the major and the minor axes of the pore.

### Example 1d

### Polymer Degradation Test In Vitro

The polymer degradation was assessed in vitro by placing a known amount of polymer in PBS (10 mg polymer/ml PBS) or in fetal bovine serum-containing PBS (10% FBS in PBS; 10 mg polymer/ml solution) at 37° C. for 1 to 60 days. The concentration of lysine liberated from the polymer was detected by the ninhydrin colorimetric reaction. See Beckwith, A. C., Paulis, J. W., and Wall, J. S. Direct estimation of lysine in corn meals by the ninhydrin color reaction. J. Agric. Food Chem. 23, 194-196, 1975. The changes in pH due to polymer degradation were assessed in parallel samples with the use of a pH meter (φ 340 pH/Temp Meter; Beckman Coulter Inc.).

Ethanol, one of the degradation products of the polymer, was monitored by gas chromatography as described by Christmore et al. Christmore, D., Kelly, R. C., and Doshier, L. Improved recovery and stability of ethanol in automated headspace analysis. J. Forensi Sci. 29, 1038-1044, 1984. The gas chromatograph (GC) was an HP 5890 series II gas chromatograph with a FID detector; equipped with HP 19395A Headspace Sampler. The GC column was a 60/80 Carbopack B, 5% Carbowax 20, and 6 feet×¼-inch OD glass-packed column. The GC oven temperature was initially 65° C. for 6.5 minutes, ramping at 20° C./min to a final temperature of 140° C. and held for 2 minutes at this temperature. The GC had an injection temperature of 150° C. and a detector temperature of 170° C.

Glycerol was assessed according to the method described by Hellmèr et al. Hellmèr, J., Arner, P., and Arner, L. Automatic luminometeric kinetic assay of glycerol for lipolysis studies. Anal. Biochem. 177, 132-137, 1989. Briefly, 0.6 ml of Tris-HCl buffer (pH 8.0), 0.2 ml of ATP monitoring agent (10 μg of firefly luciferase, 1.4×10⁻⁵ M luciferin, 10 mM magnesium acetate in 1 ml of 100 mM Tris-HCl, pH 8.0), 20 μg of glycerokinase and 0.01 mM ATP standard were mixed and measured as blank. Subsequently, 0.2 ml of sample or glycerol standard was added to the reaction mixture and luminescence measured in a luminometer (EG & G Berthold LB 9501). The concentration of glycerol in the polymer degradation products was calculated against the glycerol standard curve.

Ascorbic acid was determined according to the method described by Grudpan et al. See Grudpan, K., Kamfoo, K., and Jakmunee, J. Flow injection spectrophotometric or conductometric determination of ascorbic acid in a vitamin C tablet using permanganate or ammonia. Talanta, 49, 1023-1026, 1999. Briefly, standard/sample solutions of ascorbic acid were reacted with potassium permanganate in sulfuric acid solution, the absorbance at 525 nm, together with an ascorbic acid standard curve, were used to calculated the concentration of ascorbic acid.

The samples obtained from FBS-containing PBS were treated with an equal volume of 10% meta-phosphoric acid containing 2 mM EDTA. The formed precipitate was spun down by centrifugation (16,000 g, 2 min) and the degradation products analyzed as described above.

Mass loss was also measured using a METTLER A100 microbalance (range of 0 to 100 gram, error of ±0.1 mg). Before testing, the polymer was dried in a vacuum oven at room temperature at least 24 hr until a constant weight was obtained. The mass loss was calculated by comparing the initial mass (W₀) with that at a given time point (Wₜ), as shown in the equation below. Three individual experiments were performed for the degradation test. The results were presented as the mean±standard deviation (n=3).

Mass loss=(W₀−Wₜ)/W₀×100%

### Example 1e

### Isolation and Culture of Green Fluorescent Protein-Transgenic Mouse Bone Marrow Cells (GFP-MBMC)

Green fluorescent protein-transgenic mouse bone marrow cells (GFP-MBMC) were obtained from adult male C57 BL/6-TgN (ACTbEGFP) 10sb mice (Jackson Labs, Bar Harbor, Me.). After euthanasia by intracardial injection of Pentabarbitol, a femur was excised aseptically, cleaned, and washed in tissue culture medium (Dulbecco's Modified Eagle Medium, containing 2 mM glutamine, 10% fetal calf serum, penicillin [100 μg/mL], and streptomycin [100 μg/mL]). Subsequently, its metaphysical end was removed and the marrow flushed with 5 ml tissue culture medium containing 10 unit/ml heparin. The cells harvested were diluted in tissue culture medium, washed twice by centrifugation at 1,100×g for 10 min, and cultured in tissue culture medium containing 5 unit/ml heparin at 37° C. See Bruder, S. P., Jaiswal, N., and Haynesworth, S. E. Growth kinetics, self-renewal and the osteogenic potential of purified human mesenchymal stem cells during extensive subcultivation and following cryopreservation. J. Cell Biochem. 64, 278-294, 1997.

### Example 1f

### Culture of GFP-MBMC in Four Conditions

Green fluorescent protein-transgenic mouse bone marrow cells (GFP-MBMC) were plated at a density of 4.8×10⁴ cells/ml in Dulbecco's Modified Eagle Medium (DMEM) with 10% fetal calf serum, penicillin (100 U/ml), and streptomycin (100 μg/m1) supplemented with 30 μg/m1 of ascorbic acid (Group 1) or without ascorbic acid (Group 2). The LDI-glycerol-PEG scaffold (20.0±3 mg/piece) was washed 5 times each with 75% alcohol, sterile water, and phosphate buffer saline (1×PBS). Then the scaffold was left in cell culture medium over night. A total of 100 ial of AA-free medium containing 9.6×10⁴ cells (GFP-MBMC) was placed on each piece of scaffold in a 6-well tissue culture plate (each piece/well) and left undisturbed in an incubator for 4 h to allow the cells to adhere. Subsequently, 1.9 ml of AA-free medium was gradually added in each well (Group 3) prior to replacing the cells in the incubator (37° C., with 5% CO₂ and 95% air). The cells were replenished with fresh medium every 3 days. Similarly, LDI-glycerol-PEG-AA scaffold was treated as LDI-glycerol-PEG scaffold and used for GFP-MBMC culture (Group 4).

Visualization of GFP-MBMC under the four previously defined sets of culture conditions (Groups 1-4) was performed with a Nikon Diaphot inverted fluorescent microscope with filters DM 430 (EX380-425 and BA 510).

### Example 1g

### Cell Proliferation Assay

Cell proliferation was measured at designated times (1, 3, 5, 11 and 14 days) with a modified crystal violet dye-binding assay. See Andreoni, G., Angeretti, N., Lucca, E., and Forloni, G. Densitometric quantification of neuronal viability by computerized image analysis. Exp. Neurol., 148, 281-287, 1997. Cells cultured under the four sets of conditions set forth above were rinsed with Tyrode's balanced salt solution and fixed for 15 min in 1% (v/v) buffered glutaradehyde. The fixed cells were rinsed twice with distilled water and air-dried. The dried cultures were stained for 30 min with 0.1% crystal violet (w/v) in distilled water. The crystal violet was extracted from the cells by a 4-h incubation at room temperature in 1% Triton X-100. Triton extracts were measured at 600 nm on a micro plate reader. Absorbance values were converted into cell numbers extrapolated from established standard curves.

### Example 1h

### Alkaline Phosphatase (ALP) Assay

Alkaline phosphatase (ALP) secretion was assayed in both the cells and media of four cell-culture conditions at designated times (1, 3, 5, 11 and 14 days) by the method of Ishaug et al. Ishaug, S. L., Crane, G. M., Miller, M. J., Yasko, A. W., Yaszemski, M. J., and Mikos, A. G. Bone formation by three-dimensional stromal osteoblast culture in biodegradable polymer scaffolds. J. Biomed. Mater. Res., 36, 17-28, 1997. The medium was collected at the appropriate time point following an initial seeding of 9.6×10⁴ cells/well in a 6-well plate with or without scaffold. At the end of the experimental period, GFP-MBMC-seeded scaffolds were washed with PBS and then frozen. Upon thawing, the scaffold was homogenized with 1 ml Tris buffer (pH 8.0). Aliquots of 20 μl were incubated with 1 ml of a p-nitrophenyl phosphate solution (16 mmol/l, Diagnostic Kit 245, Sigma) at 30° C. for up to 5 min. Enzyme activity was calculated after measuring the absorbance of p-nitrophenol product formed at 405 nm on a microplate reader, and compared with serially diluted standards. The cells grown in the tissue culture plate without scaffold (Groups 1 and 2) were washed with phosphate buffered saline, and ALP activity of the cell lysates was measured as described above.

### Example 1i

### Histochemical Staining-Alkaline Phosphatase Activity and in Vitro Mineralization

The cells were cultured for two weeks and rinsed three times in PBS and fixed in 95% (v/v) ethanol and stained using an alkaline phosphatase kit (Sigma kit no. 86) according to the manufacture's instructions. Colonies were determined to be alkaline phosphatase-positive if any cells showed observable staining by light microscopy. Cultures were analyzed histologically for mineral deposition by staining with silver nitrate (1% w/n) for 60 minutes in bright sunlight according to the von Kossa method. See Sheehan, D., and Hrapchak, B. Theory and practice of histotechnology. 2^{nd} Ed. Battelle Press, Ohio, 1980, pp 226-227.

### Example 1j

### Determination of Collagen Type I Production in GFP-MBMC Grown in Four Conditions

Sirius Red F3B (Sigma) was used to examine the collagen type I synthesis in GFP-MBMC grown under the four sets of culture conditions. The dye was dissolved in saturated aqueous picric acid at a concentration of 1 mg/ml. Bouin's fluids (for cell fixation) were prepared by mixing 15 ml saturated aqueous picric acid with 5 ml 35% formaldehyde and 1 ml glacial acetic acid. Freshly prepared dye solution was used for each experiment. The cells were washed with PBS before they were fixed with 1 ml Bouin's fluids for 1 h. The fixation solution was removed by suction and the cells were washed in running tap water for 15 min. The culture dishes were air dried before adding 1 ml Sirius Red dye reagent following the routine protocol. Jundt G, T-R H. In situ measurement of collagen synthesis by human bone cells with a Sirius Red-based colorimetric microassay: effects of transforming growth factor β2 and ascorbic acid 2-phosphate. Histochem. Cell Biol. 11, 271-276, 1999 The cells were stained for 1 h under mild shaking on a micro plate shaker. Thereafter, the dye solution was removed by suction, and the cells were washed with 0.01 N hydrochloric acid to remove all non-bound dye. The cell morphology was photographed before dissolving the stain. The stained material was dissolved in 0.2-0.3 ml 0.1 N sodium hydroxide using a microplate shaker for 30 min at room temperature. The dye solution was transferred to 96-well microplates and the optical density (OD) measured at 550 nm using 0.1 N sodium hydroxide as a blank.

Soluble calf skin collagen type I was used for the standard curves run with each assay (see FIG. 14). Standard deviations in quadruplicates did not exceed 5%. Three individual experiments were performed for type I collagen. The results were presented as the mean±standard deviation (n=3).

### Example 1k

### Comparison of mRNA Expression for Collagen Type I and Transforming Growth Factor β₁ (TGF-β₁) in GFP-MBMC Cultured Under Four Conditions

After the culture of GFP-MBMC under the four sets of culture conditions, the cells were briefly washed with PBS, and RNA was extracted with the use of RNA extraction kit (Qiagen Inc., Santa Clara, Calif.). A total of 1 μg of RNA was mixed with 2 μg oligo dT (12-18 oligomer; Perkin Elmer, Norwalk Conn.) in reverse transcription buffer and incubated for 10 min at room temperature. Thereafter, the reaction mixture was cooled on ice and incubated with 200 unit of M-MLV reverse transcriptase for 60 min at 37° C. The cDNA, thus obtained, was amplified with 0.1 μg of specific primers in a reaction mixture containing 200 μM dNTP, and 0.1 units of Taq polymerase in PCR buffer (Perkin Elmer, Norwalk Conn.). PCR was performed in a cDNA thermal cyvle (Perkin Elmer, Norwalk Conn.) for 30 cycles of 40 s denaturation at 94° C., 40 s annealed at 62° C., and 60 s extended at 72° C. Because it was found that the glomerular expression of glyceraldehydes-3-phosphate dehydrogenase (GAPDH) remained stable in the mice, GAPDH was used as a housekeeping gene control. The primers used were 5′-CCATGGAGAAGGCCGGGG-3′ (sense) and 5′-CAAAGTTGTCATGGATGACC-3′ (antisense) for mouse GAPDH; 5′-GTGAACCTGGCAAACAAGGT-3′ (sense) and 5′-CTGGAGACCAGAGAAGCCAC-3′ (anti sense) for α-type I collagen; 5′-GGCTTCTAGTGCTGACG-3′ (sense) and 5′-GGGTGCTGTTGTACAAAG-3′ (antisense) for transforming growth factor β₁. Wang, S. N., Lanine, J., and Hirschberg, R. Role of glomerular ultrafiltration of growth factors in progressive intertitial fibrosis in diabetic nephropathy. Kindney International, 57, 1002-1014, 2000; Zheng, F., Fornoni, A., Elliot, S. J., Guan, Y., Breyer, M. D., Striker, L. J., and Striker. G. E. Upregulation of type I collagen by TGF-β₁ in mesangial cells is blocked by PPARγ activation. Am. J. Physiol. Renal. Physiol. 282, F639-F648, 2002; Derynck, R., Jarrett, J. A., Chen, E. Y., and Goeddel, D. V. The murine transforming growth factor-beta precursor. J. Biol. Chem. 261, 4377-4379, 1986. The amplification reaction products were resolved on 2.5% NuSieve agarose/TBE gels (FMC Bio-products), electrophoresed at 85 mV for 90 min, and visualized by ethidium bromide. Base ladders of 50 bp and 100 bp (Boehring Mannheim, Inc.) were included as standards.

### Example 1l

### Western Blot Analysis for Type I Collagen of GFP-MBMC Grown in Four Conditions

After two weeks of culture, the cells were harvested with Trypsin, subjected to a centrifuge and washed once with PBS. Thereafter, the lysis buffer/CLAP solution was added to the cells (Lysis buffer: 0.187 g HEPE, 0.4235 g NaCl, 0.001 g MgCl₂ and 0.19 g EGTA dissolved in 50 ml PBS. CLAP solution: 4 μl each of chymostatin, leupeptin, antipain and pepstatin A in 100 μl PBS. Lysis buffer/CLAP solution: 100 μl CLAP solution added to 6.6 ml Lysis buffer). To assure complete rupture of the cells, the tubes were stored at −20° C. for 12 hrs. Total protein concentration was quantified using the BCA protein assay kit (Fisher Scientific, USA), which measured the light absorbance at 562 nm verses a standard curve on a microplate reader.

The protein obtained as described above was re-suspended in Laemmli sample buffer, followed by protein separation on an SDS-10% polyacrylamide gel. Western blotting was performed as described previously. See Laemmli, U.K. Cleavage of structural proteins during the assembly of the head of bacteriophage T4. Nature 227, 680-685, 1970; and Zhang, J., Sagara, Y., Fontana, M., Dupre, S., Cavallini, D., and Kodama, H. Effect of cystathionine and cystathionine metabolites on the phosphorylation of tyrosine residues in human neutrophils. Biochem. Biophys. Res. Commun 224, 849-854, 1996.

To ensure that equal amount of total protein was loaded to the membrane, GAPDH (glycerealdehyde-3-phosphate dehydrogenase) was detected using rat-anti-mouse GAPDH antibody (ICN Biochemicals, USA) followed by peroxidase-conjugated rabbit-anti-rat IgG antibody (E.Y. Laboratories, Inc. USA). Next, Western blotting membranes were prepared in the same method for detection of type I collagen using goat-anti-mouse collagen I antibody (ICN Biochemicals, USA) followed by peroxidase-conjugated rabbit-anti-goat IgG antibody (E.Y. Laboratories, Inc. USA). Molecular weights of the proteins were determined using prestained molecular weight standards (14,300-200,000 molecular weight range; GIBCO BRL). The lanes were scanned by an EPSON GT 8000 (SEIKO EPSON Co., Japan) and the intensity of the protein bands were analysized using NIH Image software (Wayne Rasband, National Institute of Health, USA).

### Example 1m

### Statistical Analysis

Data presented herein are the result of three separate experiments performed in cell cultures and mRNA expression. For biochemical data (ALP activity and hydroxyproline concentration) and degradation products assay each point represents the mean±standard deviation of three measurements of each sample. Statistical analyses included an analysis of variance model (ANOVA) and the multiple comparison test (Fisher's Least Significant Difference), with significance established at p≦0.05.

### Example 2

### LDI/PEG/Glucose/Dexamethasone Polymer and LDI/PEG/Glucose/β-Glycerol Phosphate Polymer

### Example 2a

### Synthesis of LDI-PEG-Glucose Containing Bioactive Reagents Polymer Foam

0.18 g glucose (1 mmol; —OH 5 mmol) was dissolved with 5 ml DMSO in a dry round-bottomed flask, flushed with nitrogen and then the flask was fitted with a rubber septum and sealed. Subsequently, 1 ml of LDI (5.45 mmol, —NCO 10.92 mmol) was added to the flask with a syringe. The reaction mixture was stirred in the dark at room temperature for 5 days. The formation of urethane linkage was monitored by FT-IR spectra. 2 ml of PEG (molecular weight about 400; 5.64 mmol; —OH 11.27 mmol) were added when FT-IR spectra shown 50% of isocyanate group left in the reaction mixture. The reaction mixture was stirred for another 3 days. Then another 1 ml of LDI (5.45 mmol, —NCO 10.92 mmol) was added and continued reaction for another 3 days. 0.5 ml of water with or without bioactive reagents was added, stirred for 30 min to make foam. For the dexamethasone-containing polymer foam, 5.6 μg dexamethasone (Dex) was added and the concentration of dexamethasone in the polymer foam was 1.2 μg/g foam; for the β-glycerophosphate-containing polymer foam, 162 mg β-glycerophosphate (β-GP) was added and the concentration of β-glycerophosphate in the polymer was 34.1 mg/g foam. For the ascorbic acid-containing polymer foam, 1.25 mg ascorbic acid (Vc) was added and the concentration of ascorbic acid in the polymer was 295.5 μg/g foam. For any other two or three bioactive reagents-containing polymer, the same concentration of each compound was added in each polymer to get Vc+β-GP; Vc+Dex; and Vc+β-GP+Dex polymer foams.

### Example 2b

### Cell Culture on the Bioactive Reagents-Containing Polymer Foams

Mouse bone cells (OPC) were plated at a density of 9.6×10⁴/well in Dulbecoo's modified Eagle's medium (DMEM) with 10% fetal calf serum, penicillin (100 U/ml), and streptomycin (100 μg/mL) supplemented without any bioactive reagents (Group 1), with 5 mM β-glycerophosphate (group 2); with 100 nM dexamethasone (Group 3); with 50 μM ascorbic acid (Group 4); with 50 μM ascorbic acid and 5 mM β-glycerophosphate (Group 5); with 50 μM ascorbic acid and 100 nM dexamethasone (group 6); and with 50 μM ascorbic acid and 100 nM dexamethasone and 5 mM 3-glycerophosphate (group 7) in a 6-well tissue culture plate.

The scaffold (100±10 mg/piece) was washed five times, each with 75% alcohol, sterile water, and phosphate-buffered saline (PBS). Scaffold was left in DMEM overnight. A total of 100 ml of DMEM containing 9.6×10⁴ cells was placed on each piece of scaffold in a 6-well tissue culture plate (each piece per well) and left undisturbed in an incubator for 4 hours to allow the cells to adhere. Subsequently, 2.9 ml of DMEM was gradually added to each well. The cells were cultured at 37° C., with 5% CO₂ and 95% air for four weeks. The cells were replenished with fresh medium every 3 days.

During the culture, the scaffold degraded and released bioactive reagents into the medium. For β-glycerophosphate-containing polymer foam, the concentration of β-glycerophosphata (β-GP) in each well was 5 mM when the polymer foam degraded completely. Because 162 mg β-GP was added into 4.75 g polymer foam, there was 3.41 mg β-GP (FW 216) in each piece (0.1 g) of the polymer foam. Thus, the concentration of β-GP was about 5.26 mM in each well. Similarly, in the dexamethasone-containing polymer foam, there was 0.12 βg dexamethasone (Dex; FW 392.5) in each piece of the polymer foam, so the concentration of Dex in the medium was 102 nM. There was 25.95 μg ascorbic acid (Vc; FW 173) in each piece of the ascorbic acid-containing polymer foam so the ascorbic acid concentration was 50 βM in the medium.

### Example 3

### LDI/Glycerol/DNA Polymer

Materials.

Dulbecco's Modified Eagle's Medium (DMEM) was obtained from Life Technologies (Grand Island, N.Y., USA). Molecular biology reagents were purchased from Perkin Elmer (Norwalk, Conn., USA). Poly(ethylene glycol) (PEG, average Mn 400) was obtained from Aldrich (Milwaukee, Wis., USA). 0, 0′-Bis(2-aminopropyl)-polypropylene glycol 300 (APPG, Mr 400) was purchased from Fluka Chemie AG (Buchs, Switzerland). pIRESneo-Runx2 plasmid is a 6.8 kb cDNA inserted into a pIRESneo vector (a gift from Dr. Huihua Fu, Bone Tissue Engineering Center, Carnegie Mellon University, Pittsburgh, Pa., USA). L-lysine ethyl ester di-hydrochloride, a penicillin-streptomycin solution (10,000 units penicillin and 10 μg streptomycin/ml saline) and all other reagents were analytical grad and obtained from Sigma Chemical Co. (St. Louis, Mo., USA) unless otherwise stated.

### Example 3a

### Synthesis

LDI was synthesized according to a previously described method. Zhang J, Beckman E J, Piesco N P, Agarwal S. A new peptide-based urethane polymer: synthesis, biodegradation, and potential to support cell growth in vitro. Biomaterials 2000; 21: 1247-1258. An injectable polyurethane scaffold for gene delivery was synthesized by a one-step injection reaction. In a typical experiment, PEG and APPG were sterilized by a 0.2 μm Millipore filter and mixed in a dry flask, subsequently, filter sterilized suspension of plasmid DNA containing the reporter gene LacZ or Runx2 was added into the mixture of PEG and APPG, mixed well, and labeled as solution I. Then, solution I and filter sterilized LDI (the molar rate of PEG:APPG:LDI=1:1:2.2) was added slowly into a design model with two syringes. A DNA plasmid-containing urethane polymer was obtained in 10 min.

### Example 3b

### Analyses of DNA Plasmid Release Kinetics

To measure the release kinetics of Runx2 plasmid from carrier matrix, the LDI-PEG-APPG matrix containing Runx2 was created under wet and dry conditions. Wet polymer was synthesized by reacting a solution of Runx2 plasmid in PEG-APPG with LDI and letting the mixture foam for 30 min. Dry polymer was synthesized by first making the PEG-APPG-LDI matrix, and allowing it to set for 24 hrs to dry prior to addition of Runx2 plasmid to the matrix. Each piece of wet or dry polymer weighted 100 mg and contained 10 μg of Runx2 plasmid. For control purposes, water was used in the place of Runx2, and PEG-APPG-LDI carrier without the plasmid was synthesized. Each sample was immersed in 1 ml of phosphate-buffered saline (PBS) and incubated under physiological conditions for 60 days. The buffer was changed daily, and analyzed spectrophotometrically at 260 nm to monitor DNA release. When analyzing data, the absorption values of the control group were subtracted from the values of the experimental samples.

### Example 3c

### Transfection Efficiency of a Reporter Gene (LacZ)

LacZ plasmid solution was added into adequate amounts of PEG (0.25 ml), APPG (0.3 ml) and LDI (0.3 ml) to make LDI-PEG-APPG-LacZ scaffold. NIH 3T3 cells were plated on the LDI-PEG-APPG-LacZ (0 or 10 μg LacZ/piece polymer; 100 mg/piece) polymer scaffold (wet) at a concentration of 2×10⁶ cells/well/piece polymer in 2 ml DMEM containing 5% fetal bovine serum and incubated for 48 hours. Control group 1 was cultured on a polystyrene tissue culture plate and transfected with LacZ gene. Control group 2 was that NIH3T3 cells grown in tissue culture plate transfected LacZ gene by calcium phosphate precipitation technique. See Zheng W, Zhao Q. Establishment and characterization of an immortalized Z310 choroidal epithelial cell line from murine choroid plexus. Brain Res 2002; 958(2):371-80. The transduction efficiency of NIH 3T3 cells cultured with LDI-PEG-APPG-LacZ polymer was estimated by staining with chromogenic substrate, 5-bromo-4-chloro-3-iodolyl-beta-d-galactopyranoside (X-gal), which is the modification of a procedure described previously. See Nakayama Y, Ji-Youn K, Nishi S, Ueno H, Matsuda T. Development of high-performance stent: gelatinous photogel-coated stent that permits drug delivery and gene transfer. J Biomed Mater Res 2001; 57(4): 559-566. Briefly, the cells were fixed with phosphate-buffered saline (PBS) containing 0.5% glutaraldehyde for 15 min at room temperature. After fixation, LacZ expression was evaluated by histochemical staining with X-gal in PBS containing 5 mmol/l of K₃Fe(CN)₆, 5 mmol/l of K₄Fe(CN)₆ 3H₂O, 1 mmol/l of MgCl₂, and 1 mg/ml of X-gal at 37° C. for 6 hours. Transfection efficiency was determined by quantitating the positively stained cells in ten randomly chosen locations.

### Example 3d

### In Vitro Transfection of pIRESneo-Runx2 Plasmid

The in vitro assays were executed to verify transcription of Runx2 plasmid into the transfected cells using RT-PCR techniques. Runx2-containing polymer scaffolds were prepared as described above. Following trypsinization, NIH3T3 cells were seeded onto the LDI-PEG-APPG-pIRESneo-Runx2 scaffold (0 or 10 μg pIRESneo-Runx2 plasmid/piece polymer; 100 mg/piece) at a concentration of 2×10⁶ cells/well/piece polymer in 2 ml DMEM containing 5% fetal bovine serum and cultured at 37° C. with 5% CO₂. The medium was changed every 3 days. The transfection efficiency was evaluated at days 21 by osteogenic phenotypes including alkaline phosphatase activity, osteocalcin, procollagen type I, and Runx2 gene expressions in NIH3T3 cells.

### Example 3e

### Alkaline Phosphatase Activity

Alkaline phosphatase (ALP) secretion was assayed in NIH3T3 cells-transfected with pIRESneo-Runx2 plasmid by the method of Ishaug et al. See Ishaug S L, Crane G M, Miller M J, Yasko A W, Yaszemski M J, Mikos AG. Bone formation by three-dimensional stromal osteoblast culture in biodegradable polymer scaffolds. J Biomed Mater Res 1997; 36: 17-28. At days 21, NIH3T3-seeded scaffold were washed with PBS and then frozen. Upon thawing, the scaffold was homogenized with 1 ml Tris-HCl buffer (pH 8.0). Aliquots of 20 l were incubated with 1 ml of p-nitrophenyl phosphate solution (16 mmol/1, Diagnostic Kit 245, Sigma) at 30° C. for up to 30 min. Enzyme activity was calculated after measuring the absorbance of p-nitrophenol product formed at 405 nm on a microplate reader, and compared with serially diluted standards. The cells grown in the tissue culture plate without scaffold were washed with phosphate buffered saline, and ALP activity of the cell lysates was measured as the same as that of the cells grown on the scaffold.

### Example 3f

### Comparison of mRNA Expression for Procollagen Type I, Osteocalcin, and Runx2

Following transfection and culture of NIH3T3 cells on LDI-PEG-APPG-pIRESneo-Runx2 polymer at days 21, the cells were washed twice with PBS for 5 min each time, and their mRNA was extracted with RNA extraction kit (Qiagen Inc., Santa Clara, Calif.). A total of 1 μg of mRNA was mixed with 1 μg oligo dT (12-18 oligomer; Perkin Elmer, Norwalk Conn.) in reverse transcription buffer and incubated for 10 min at room temperature. Thereafter, the reaction mixture was cooled on ice and incubated with 200 U of M-MLV reverse transcriptase for 60 min at 37° C. The cDNA was amplified with 0.1 μg of specific primers in a reaction mixture containing 200 μM dNTP, and 0.1 units of Taq polymerase in PCR buffer (Perkin Elmer, Norwalk Conn.). Glyceraldehydes-3-phosphate dehydrogenase (GAPDH) was used as a housekeeping gene control as described above. PCR was performed in a DNA thermal cycle (Perkin Elmer, Norwalk Conn.) for 30 cycles of 35 s at 94° C., 35 s at 56° C., and 40 s at 72° C. for Runx2; 35 s at 94° C., 40 s at 56° C., and 50 s at 72° C. for procollagen type I; 35 s at 94° C., 40 s at 58° C. and 40 s at 72° C. for osteocalcin; 35 s at 94° C., 35 s at 56° C. 50 s at 72° C. for GAPDH. The amplification reaction products were resolved on 2.5% NuSieve agarose/TBE gels (FMC Bio-products), electrophoresed at 85 mV for 90 min, and visualized by ethidium bromide. Base ladder of 1 kb was included as standards.

### Example 3g

### Statistical Analysis

Three separate experiments were performed and statistical analyses were carried out on A MICROSOFT EXCEL® program. All quantitative data reported herein are expressed as mean±standard deviation of the THREE measurements of each sample. Statistical analyses included an analysis of variance model (ANOVA) and the Multiple Comparison Test (Fisher's Protected Least Significant Difference), with significance established at p≦0.05.

The foregoing description and accompanying drawings set forth preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

1. A biodegradable and biocompatible polyurethane composition synthesized by:
reacting isocyanate groups of at least one multifunctional isocyanate compound with at least one bioactive agent having at least one reactive group —X which is a hydroxyl group (—OH) or an amine group (—NH₂), the polyurethane composition being biodegradable within a living organism to biocompatible degradation products including the bioactive agent, the released bioactive agent affecting at least one of biological activity or chemical activity in the host organism.

2. The composition of claim 1 wherein the multifunction isocyanate compound is formed via conversion of amine groups of a biocompatible compound having at least two amine groups to isocyanate groups.

3. The composition of claim 2 wherein the bioactive agent has at least two reactive groups —X and —X¹ which are independently the same or different a hydroxyl group (—OH) or an amine group (—NH₂).

4. The composition of claim 3 wherein the multifunctional isocyanate compound is also reacted with at least one biocompatible polyol compound, the polyol compound having at least two reactive groups —X² and —X³ which are independently the same of different hydroxyl group (—OH) or an amine group (—NH₂).

5. The composition of claim 4 wherein the multifunctional isocyanate is also reacted with at least one biocompatible chain extender, the chain extender, wherein the chain extender is water or a compound having at least two reactive groups —X⁴ and —X⁵ which are independently the same of different hydroxyl group (—OH) or an amine group (—NH₂).

6. The composition of claim 4 wherein the multifunctional isocyanate compound, the bioactive agent and the polyol compound are reacted to form a prepolymer, the prepolymer being further reacted with at least one biocompatible chain extender, wherein the chain extender is water or a compound having at least two reactive groups —X⁴ and —X⁵ which are independently the same of different hydroxyl group (—OH) or an amine group (—NH₂).

7. The composition of claim 1 wherein the multifunctional isocyanate compound is a prepolymer formed by the reaction of a multifunctional isocyanate precursor and at least one biocompatible polyol compound, the polyol compound having at least two reactive groups —X² and —X³ which are independently the same of different hydroxyl group (—OH) or an amine group (—NH₂), the multifunction isocyanate precursor being formed via conversion of amine groups of a biocompatible compound having at least two amine groups to isocyanate groups.

8. The composition of claim 7 wherein the prepolymer is contacted with the bioactive agent.

9. The composition of claim 8 wherein the bioactive compound is in a solution with at least one biocompatible chain extender, wherein the chain extender is water or a compound having at least two reactive groups —X⁴ and —X⁵ which are independently the same of different hydroxyl group (—OH) or an amine group (—NH₂).

10. The composition of claim 1 wherein the bioactive agent has a therapeutic effect in the organism upon release.

11. The composition of claim 1 wherein the bioactive agent is an enzyme, an organic catalysts a ribozyme, an organometallic, a protein, a glycoprotein, a lipoprotein, a peptide, a polyamino acid, an antibody, a nucleic acid, a steroidal molecule, an antibiotic, an antivirals, an antimycotic, an anticancer agent, an immunosuppressant, a cytokine, a carbohydrate, an oleophobic, a lipid, an extracellular matrix, a component of an extracellular matrix, a chemotherapeutic agent, an anti-rejection agent, an analgesic agent, an anti-inflammatory agent, a hormone, a virus, a viral vector, a vireno, or a prion.

12. The composition of claim 7 where the multifunctional isocyanate precursor is an aliphatic multifunctional isocyanate.

13. The composition of claim 7 where the multifunctional isocyanate precursor is derived from a biomolecule.

14. The composition of claim 13 where the multifunctional isocyanate precursor is derived from an amino acid.

15. The composition of claim 7 where the polyol compound is a biomolecule.

16. The composition of claim 15 where the polyol compound is a hydroxylated biomolecule.

17. The composition of claim 9 where the chain extender is a biomolecule.

18. The composition of claim 9 where the chain extender is water.

19.-39. (canceled)

40. A method of synthesizing a bone tissue engineering scaffold including the steps of:
coating a biodegradable and bioactive polyurethane polymer with human osteoblastic precursor cells, the polymer being synthesized by reacting isocyanate groups of at least one multifunctional isocyanate compound with at least one bioactive agent having at least one reactive group —X which is a hydroxyl group (—OH) or an amine group (—NH₂), the polyurethane being biodegradable within a living organism to biocompatible degradation products including the bioactive agent, the released bioactive agent affecting at least one of biological activity or chemical activity in the host organism; and
culturing the osteoblastic precursor cells under conditions suitable to promote cell growth.

41.-68. (canceled)

69. An implant for insertion into an organism, the implant being formed external to the organism and subsequently placed into the organism, the implant being formed by reacting isocyanate groups of at least one multifunctional isocyanate compound with at least one bioactive agent having at least one reactive group —X which is a hydroxyl group (—OH) or an amine group (—NH₂), the polyurethane composition being biodegradable within a living organism to biocompatible degradation products including the bioactive agent, the released bioactive agent affecting at least one of biological activity or chemical activity in the host organism.

70.-103. (canceled)
